(19) United States
(12) Patent Application Publication
(10) Pub. No.: US 2011/0312934 A1
(43) Pub. Date: Dec. 22, 2011

(54) 2-ALKYL-6-CYCLOAMINO-3-(PYRIDIN-4-YL)IMIDAZO[1,2-B]-PYRIDAZINE DERIVATIVES, PREPARATION THEREOF, AND THERAPEUTIC APPLICATION THEREOF

(75) Inventors: Antonio Almario Garcia, Paris (FR); Philippe Burnier, Paris (FR); Sylvain Cote-Des Combes, Paris (FR); Jean-François Gibert, Paris (FR); Christophe Pacaud, Paris (FR); Frédéric Puech, Paris (FR); Yulin Chiang, Bridgewater, NJ (US); Larry Davis, Bridgewater, NJ (US); Zhongli Gao, Bridgewater, NJ (US); Qiuxia Zhao, Bridgewater, NJ (US)

(73) Assignee: SANOFI-AVENTIS, Paris (FR)

(21) Appl. No.: 13/058,665

(22) PCT Filed: Aug. 12, 2009
(86) PCT No.: PCT/FR09/01001

§ 371 Date: Sep. 2, 2011

Related U.S. Application Data
(60) Provisional application No. 61/088,126, filed on Aug. 12, 2008.

(30) Foreign Application Priority Data
Aug. 12, 2008 (FR) 0804573

(51) Int. Cl. A61K 31/551; C07D 498/10; C07D 471/08; A61P 25/30; A61K 31/5386; A61P 35/00; A61P 25/00; C07D 487/04; A61K 31/5025

(52) U.S. Cl. 514/210.21; 544/236; 544/71; 540/569; 514/248; 514/233.2; 514/221

(57) ABSTRACT

The invention relates to 2-alkyl-6-cycloamino-3-(pyridin-4-yl)imidazo[1,2-b]pyridazine derivatives of the general formula (I) where: R₂ is a C₁₋₆-alkyl, C₃₋₇-cycloalkyl, C₃₋₇-cycloalkyl-C₁₋₄-alkyl, C₁₋₄-alkyloxy-C-M-alkyl, C₃₋₇-cycloalkyloxy-C₁₋₄-alkyl>C₃₋₇-cycloalkyl-C₁₋₄-alkyloxy-C₁₋₄-alkyl, hydroxy-C₁₋₆-alkyl, C₁₋₄-fluoroalkyl group; R₃ is a hydrogen atom or a substituent selected from halogen atoms and the C₁₋₃ alkyl, —NR₄R₅, hydroxyl or C₁₋₄ alkyloxy groups; A is a C₁₋₇-alkylene group optionally substituted by one or two Rₐ groups; B is a C₁₋₇-alkylene group optionally substituted by one or two R_{b} groups; L is either a nitrogen atom optionally substituted by an R_{c} or R_{d} group or a carbon atom substituted by an Rₑ₁ group and an R_{d} group or by two Rₑ₂ groups; Rd is a group selected from a hydrogen atom or a C₁₋₆-alkyl, C₃₋₇-cycloalkyl, C₃₋₇-cycloalkyl-C₁₋₆-alkyl, C₁₋₆-alkylthio-C₁₋₆-alkyl, C₁₋₆-alkyloxy-C₁₋₆-alkyl, C₁₋₆-fluoroalkyl, hydroxy-C₁₋₆-alkyl group; R_{f} is a C₁₋₆-alkyl, C₃₋₇-cycloalkyl, C₃₋₇-cycloalkyl-C₁₋₆-alkyl, C₁₋₆-alkyloxy-C₁₋₆-alkyl, C₃₋₇-cycloalkyloxy-C₁₋₄-alkyl, C₃₋₇-cycloalkyl-C₁₋₄-alkyloxy-C₁₋₄-alkyl, hydroxy-C₁₋₆-allyl, C₁₋₆-fluoroalkyl or benzyl group. The invention also relates to a method for preparing same and to the therapeutic application thereof.

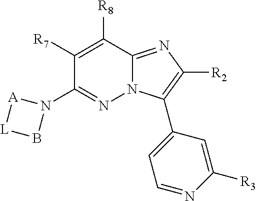

The present invention relates to 2-alkyl-6-cycloamino-3-(pyridin-4-yl)imidazo[1,2-b]pyridazine derivatives, to their preparation and to their therapeutic use in the treatment or prevention of diseases involving casein kinase 1 epsilon and/or casein kinase 1 delta.

The present invention provides the compounds conforming to the general formula (I)

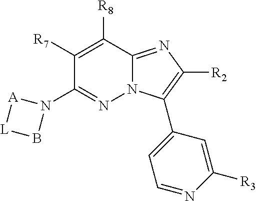

in which
R₂ represents a C₁₋₆-alkyl, C₃₋₇-cycloalkyl, C₃₋₇-cycloalkyl-C₁₋₄-alkyl, C₁₋₄-alkyloxy-C₁₋₄-alkyl, C₃₋₇-cycloalkyloxy-C₁₋₄-alkyl, C₃₋₇-cycloalkyl-C₁₋₄-alkyloxy-C₁₋₄-alkyl, hydroxy-C₁₋₆-alkyl or C₁₋₄-fluoroalkyl group;
R₃ represents a hydrogen atom or a substituent selected from halogen atoms and C₁₋₃-alkyl, —NR₄R₅, hydroxyl or C₁₋₄-alkyloxy groups;
A represents a C₁₋₇-alkylene group optionally substituted by one or two groups Rₐ;
B represents a C₁₋₇-alkylene group optionally substituted by a group R_{b};
L represents either a nitrogen atom optionally substituted by a group R_{c} or R_{d}, or a carbon atom substituted by a group Rₑ₁ and a group R_{d} or two groups Rₑ₂;
   the carbon atoms of A and of B being optionally substituted by one or more groups R_{f} that are identical or different from one another;
   Rₐ, R_{b} and R_{c} are defined such that:
two groups Rₐ may together form a C₁₋₆-alkylene group;
Rₐ and R_{b} may together form a bond or a C₁₋₆-alkylene group;
Rₐ and R_{c} may together form a bond or a C₁₋₆-alkylene group;
R_{b} and R_{c} may together form a bond or a C₁₋₆-alkylene group;
R_{d} represents a group selected from the hydrogen atom and C₁₋₆-alkyl, C₃₋₇-cycloalkyl, C₃₋₇-cycloalkyl-C₁₋₆-alkyl, C₁₋₆-alkylthio-C₁₋₆-alkyl, C₁₋₆-alkyloxy-C₁₋₆-alkyl, C₁₋₆-fluoroalkyl and hydroxy-C₁₋₆-alkyl groups;
Rₑ₁ represents a group —NR₄R₅ or a cyclic monoamine optionally containing an oxygen atom, the cyclic monoamine being optionally substituted by one or more substituents selected from the fluorine atom and C₁₋₆-alkyl, C₁₋₆-alkyloxy and hydroxyl groups;
two radicals Rₑ₂ form, with the carbon atom which carries them, a cyclic monoamine optionally containing an oxygen atom, this cyclic monoamine being optionally substituted by one or more groups R_{f} that are identical or different from one another;
R_{f} represents a C₁₋₆-alkyl, C₃₋₇-cycloalkyl, C₃₋₇-cycloalkyl-C₁₋₆-alkyl, C₁₋₆-alkyloxy-C₁₋₆-alkyl, C₃₋₇-cycloalkyloxy-C₁₋₄-alkyl, C₃₋₇-cycloalkyl-C₁₋₄-alkyloxy-C₁₋₄-alkyl, hydroxy-C₁₋₆-alkyl or C₁₋₆-fluoroalkyl group;
R₄ and R₅ represent, independently of one another, a hydrogen atom or a C₁₋₄ alkyl, C₃₋₇-cycloalkyl or C₃₋₇-cycloalkyl-C₁₋₆-alkyl group;
R₇ and R₈ represent, independently of one another, a hydrogen atom or a C₁₋₆-alkyl group.

The compounds of formula (I) may include one or more asymmetric carbon atoms. They may therefore exist in the form of enantiomers or diastereoisomers. These enantiomers and diastereoisomers, and also mixtures thereof, including the racemic mixtures, form part of the invention.

The compounds of formula (I) may exist in the form of bases or acid addition salts. Such addition salts form part of the invention. These salts are advantageously prepared with pharmaceutically acceptable acids, although the salts of other acids that are useful, for example, for purifying or isolating compounds of formula (I) likewise form part of the invention.

The compounds of formula (I) may also exist in the form of hydrates or solvates, in other words in the form of associations or combinations with one or more molecules of water or with a solvent. Such hydrates and solvates likewise form part of the invention.

In the context of the invention the following definitions apply:
C_{t-z}, where t and z may adopt the values from 1 to 7: a carbon chain that can have from t to z carbon atoms; for example, C₁₋₇ is a carbon chain which may have from 1 to 7 carbon atoms;
alkyl: a linear or branched, saturated aliphatic group; for example, a C₁₋₆-alkyl group represents a linear or branched carbon chain of 1 to 6 carbon atoms, for example a methyl, ethyl, propyl, isopropyl, butyl, isobutyl, tert-butyl, pentyl or hexyl;
alkylene: a linear or branched, saturated divalent alkyl group; for example a C₁₋₆-alkylene group represents a linear or branched divalent carbon chain of 1 to 6 carbon atoms, for example a methylene, ethylene, 1-methylethylene or propylene;
cycloalkyl: a cyclic alkyl group; for example, a C₃₋₇-cycloalkyl group represents a cyclic carbon group of 3 to 7 carbon atoms, for example a cyclopropyl, cyclobutyl, cyclopentyl, cyclohexyl or cycloheptyl;
acyl: an alkyl-C(O)— group;
hydroxyl: an —OH group;
cyclic monoamine: a saturated cyclic carbon chain containing 1 nitrogen atom;
hydroxyalkyl: an alkyl group in which a hydrogen atom has been substituted by a hydroxyl group;
alkyloxy: an —O-alkyl group;
alkylthio: an —S-alkyl group;
fluoroalkyl: an alkyl group in which one or more hydrogen atoms have been substituted by a fluorine atom;
fluoroalkyloxy: an alkyloxy group in which one or more hydrogen atoms have been substituted by a fluorine atom;
a halogen atom, a fluorine, chlorine, bromine or iodine atom;
aryl: a monocyclic or bicyclic aromatic group containing between 6 and 10 carbon atoms. Examples of aryl group include the groups phenyl or naphthyl.

Non-limitative examples of cyclic amines or diamines formed by N, A, L and B include more particularly:
aziridine, azetidine, pyrrolidine, piperidine, azepine, morpholine, thiomorpholine, homopiperidine, azabicycloheptane, azabicyclooctane, azabicyclononane, azaoxobicycloheptane, azathiabicycloheptane, azaoxobicyclooctane, azathiabicyclooctane;
piperazine, homopiperazine, diazacyclooctane, diazacyclononane, diazacyclodecane, diazacycloundecane;
hexahydropyrrolopyrazine, octahydropyrrolodiazepine, hexahydropyrrolopyrrole, octahydropyrrolopyridine, decahydronaphthyridine;
diazabicycloheptane, diazabicyclooctane, diazabicyclononane;
diazaspiroheptane, diazaspirooctane, diazaspirononane, diazaspirodecane, diazaspiroundecane, oxadiazaspiroundecane.

Among the compounds of general formula (I) that are subject matter of the invention, a first group of compounds is composed of the compounds for which R₂ represents a C₁₋₄-alkyl, C₃₋₄-cycloalkyl-C₁₋₄-alkyl, C₁₋₄-alkyloxy-C₁₋₄-alkyl or C₁₋₄-fluoroalkyl group; A, L, B, R₃, R₇ and R₈ being as defined above.

Among the compounds of general formula (I) that are subject matter of the invention, a second group of compounds is composed of the compounds for which R₂ represents a methyl, ethyl, isopropyl, isobutyl, cyclopropyl, cyclobutyl, cyclopropylmethyl, methoxymethyl or trifluoromethyl group;

A, L, B, R₃, R₇ and R₈ being as defined above.

Among the compounds of general formula (I) that are subject matter of the invention, a third group of compounds is composed of the compounds for which R₃ represents hydrogen, fluorine or chlorine atom or a methyl, methylamino, —NH₂ or methoxy group;

A, L, B, R₂, R₇ and R₈ being as defined above.

Among the compounds of general formula (I) that are subject matter of the invention, a fourth group of compounds is composed of the compounds for which R₇ and R₈ represent, independently of one another, a hydrogen atom or a methyl group;

A, L, B, R₂ and R₃ being as defined above.

Among the compounds of general formula (I) that are subject matter of the invention, a fifth group of compounds is composed of the compounds for which:
A represents a C₁₋₇-alkylene group optionally substituted by one or two groups Rₐ;
B represents a C₁₋₇-alkylene group optionally substituted by a group R_{b};
L represents a nitrogen atom optionally substituted by a group R_{c} or R_{d},
   the carbon atoms of A and of B being optionally substituted by one or more groups R_{f} that are identical or different from one another;
two groups Rₐ may together form a C₁₋₆-alkylene group;
Rₐ and R_{b} may together form a bond or a C₁₋₆-alkylene group;
R^{a} and R_{c} may together form a bond or a C₁₋₆-alkylene group;
R_{b} and R_{c} may together form a bond or a C₁₋₆-alkylene group;
R_{d} represents a group selected from the hydrogen atom and C₁₋₆-alkyl, C₃₋₇-cycloalkyl, C₃₋₇-cycloalkyl-C₁₋₆-alkyl, C₁₋₆-alkylthio-C₁₋₆-alkyl, C₁₋₆-alkyloxy-C₁₋₆-alkyl, C₁₋₆-fluoroalkyl and hydroxy-C₁₋₆-alkyl groups;
R_{f} represents a C₁₋₆-alkyl, C₃₋₇-cycloalkyl, C₁₋₆-alkyloxy-C₁₋₆-alkyl, C₃₋₇-cycloalkyloxy-C₁₋₄-alkyl, C₃₋₇-cycloalkyl-C₁₋₄-alkyloxy-C₁₋₄-alkyl or hydroxy-C₁₋₆-alkyl group;
R₂, R₃, R₇ and R₈ being as defined above.

Among the compounds of general formula (I) that are subject matter of the invention, a sixth group of compounds is composed of the compounds for which:

the cyclic amine formed by —N-A-L-B— represents a piperazinyl, hexahydropyrrolopyrazinyl, diazabicycloheptyl, diazabicyclononyl, hexahydropyrrolopyrrole or octahydropyrrolopyridine group optionally substituted by one or more methyl, ethyl, isopropyl, cyclobutyl and/or hydroxymethyl groups;
R₂, R₃, R₇ and R₈ being as defined above.

Among the compounds of general formula (I) that are subject matter of the invention, a seventh group of compounds is composed of the compounds for which:

the cyclic amine formed by —N-A-L-B— represents a piperazin-1-yl, (R,S)-3-methylpiperazin-1-yl, (R)-3-methylpiperazin-1-yl, (S)-3-methylpiperazin-1-yl, 4-methylpiperazin-1-yl, 4-ethyl-piperazin-1-yl, 4-(isopropyl)piperazin-1-yl, 4-(cyclobutyl)piperazin-1-yl, (R,S)-3-(hydroxymethyl)piperazin-1-yl, 3,3-dimethylpiperazin-1-yl, cis-3,5-dimethylpiperazin-1-yl, (S)-hexahydropyrrolo[1,2-a]pyrazin-2-yl, (1S,4S)-2,5-diazabicyclo[2.2.1]hept-2-yl, (R,S)-2,5-diazabicyclo[2.2.1]hept-2-yl, (R,S)-1,4-diazabicyclonon-4-yl, (R,S)-hexahydropyrrolo[3,4-b]-pyrrol-5(1H)-yl, hexahydropyrrolo[3,4-c]pyrrol-2(1H)-yl, 5-methyl-hexahydropyrrolo[3,4-c]-pyrrol-2(1H)-yl, 5-isopropyl-hexahydropyrrolo[3,4-c]pyrrol-2(1H)-yl, (R,S)-octahydropyrrolo-[3,4-b]pyridin-6-yl group;
R₂, R₃, R₇ and R₈ being as defined above.

Among the compounds of general formula (I) that are subject matter of the invention, an eighth group of compounds is composed of the compounds for which:
A represents a C₁₋₇-alkylene group optionally substituted by one or two groups Rₐ;
B represents a C₁₋₇-alkylene group optionally substituted by a group R_{b};
L represents a carbon atom substituted by two groups Rₑ₂; the carbon atoms of A and of B being optionally substituted by one or more groups R_{f} that are identical or different from one another;
two radicals Rₑ₂ form, with the carbon atom which carries them, a cyclic monoamine optionally containing an oxygen atom, this cyclic monoamine being optionally substituted by one or more groups R_{f} that are identical or different from one another;
R_{f} represents a C₁₋₆-alkyl group;
R₂, R₃, R₇, and R₈ being as defined above.

Among the compounds of general formula (I) that are subject matter of the invention, a ninth group of compounds is composed of the compounds for which:

the cyclic amine formed by —N-A-L-B— represents a diazaspirononyl, diazaspirodecyl, diazaspiroundecyl or oxadiazaspiroundecyl group;
R₂, R₃, R₇ and R₈ being as defined above.

Among the compounds of general formula (I) that are subject matter of the invention, a tenth group of compounds is composed of the compounds for which:

the cyclic amine formed by —N-A-L-B— represents a 2,7-diazaspiro[3.5]non-7-yl, (R,S)-diazaspiro[4.5]dec-2-yl, 2,9-diazaspiro[5.5]undec-9-yl or 1-oxa-4,9-diazaspiroundec-9-yl;
R₂, R₃, R₇ and R₈ being as defined above.

Among the compounds of general formula (I) that are subject matter of the invention, an eleventh group of compounds is composed of the compounds for which:
A represents a C₁₋₇-alkylene group;
B represents a C₁₋₇-alkylene group;
L represents a carbon atom substituted by a group Rₑ₁ and a group R_{d};
R_{d} represents a hydrogen atom;
Rₑ₁ represents a group —NR₄R₅ in which R₄ and R₅ represent, independently of one another, a hydrogen atom or a C₁₋₄-alkyl group, or else Rₑ₁ represents a cyclic monoamine optionally containing an oxygen atom, the cyclic monoamine being optionally substituted by one or more substituents selected from C₁₋₆-alkyl and hydroxyl groups;
R₂, R₃, R₇ and R₈ being as defined above.

Among the compounds of general formula (I) that are subject matter of the invention, a twelfth group of compounds is composed of the compounds for which:
A represents a —C₂H₄₋group or a —CH₂— group;
B represents a —C₂H₄— group;
L represents a carbon atom substituted by a group Rₑ₁ and a group R_{d};
R_{d} represents a hydrogen atom;
Rₑ₁ represents a pyrrolidinyl group;
R₂, R₃, R₇ and R₈ being as defined above.

Among the compounds of general formula (I) that are subject matter of the invention, a thirteenth group of compounds is composed of the compounds for which:
the cyclic amine formed by ═N-A-L-B— represents a 4-(pyrrolidin-1-yl)-piperidin-1-yl or (R,S)-[1,3′]bipyrrolidinyl-1′-yl;
R₂, R₃, R₇ and R₈ being as defined above.

Among the compounds of general formula (I) that are subject matter of the invention, a fourteenth group of compounds is composed of the compounds for which:
R₂ represents a methyl, ethyl, isopropyl, isobutyl, cyclopropyl, cyclobutyl, cyclopropylmethyl, methoxymethyl or trifluoromethyl group;
R₃ represents hydrogen, fluorine or chlorine atom or a methyl, methylamino, —NH₂ or methoxy group;
the cyclic amine formed by —N-A-L-B— represents a piperazin-1-yl, (R,S)-3-methylpiperazin-1-yl, (R)-3-methylpiperazin-1-yl, (S)-3-methylpiperazin-1-yl, 4-methylpiperazin-1-yl, 4-ethylpiperazin-1-yl, 4-(isopropyl)piperazin-1-yl, 4-(cyclobutyl)piperazin-1-yl, (R,S)-3-(hydroxymethyl)piperazin-1-yl, 3,3-dimethylpiperazin-1-yl, cis-3,5-dimethylpiperazin-1-yl, (S)-hexahydropyrrolo[1,2-a]pyrazin-2-yl, (1S,4S)-2,5-diazabicyclo[2.2.1]hept-2-yl, (R,S)-2,5-diazabicyclo[2.2.1]hept-2-yl, (R,S)-1,4-diazabicyclonon-4-yl, (R,S)-hexahydropyrrolo[3,4-b]-pyrrol-5(1H)-yl, hexahydropyrrolo[3,4-c]pyrrol-2(1H)-yl, 5-methylhexahydropyrrolo[3,4-c]-pyrrol-2(1H)-yl, 5-isopropylhexahydropyrrolo[3,4-c]pyrrol-2(1H)-yl or (R,S)-octahydropyrrolo-[3,4-b]pyridin-6-yl group;
R₇ and R₈ represent, independently of one another, a hydrogen atom or a methyl group.

Among the compounds of general formula (I) that are subject matter of the invention, a fifteenth group of compounds is composed of the compounds for which:
R₂ represents a methyl group;
R₃ represents hydrogen atom;
the cyclic amine formed by —N-A-L-B— represents a 2,7-diazaspiro[3.5]non-7-yl, (R,S)-diazaspiro[4.5]dec-2-yl, 2,9-diazaspiro[5.5]undec-9-yl or 1-oxa-4,9-diazaspiroundec-9-yl;
R₇ and R₈ represent, independently of one another, a hydrogen atom.

Among the compounds of general formula (I) that are subject matter of the invention, a sixteenth group of compounds is composed of the compounds for which:
R₂ represents a methyl group;
R₃ represents hydrogen atom or a methyl group;
the cyclic amine formed by —N-A-L-B— represents a 4-(pyrrolidin-1-yl)piperidin-1-yl or a (R,S)-[1,3′]bipyrrolidinyl-1′-yl;
R₇ and R₈ represent, independently of one another, a hydrogen atom.

Among the compounds of general formula (I) that are subject matter of the invention, mention may be made more particularly of the following compounds:
2-Methyl-6-piperazin-1-yl-3-pyridin-4-yl-imidazo[1,2-b]pyridazine and its hydrochloride (3:1);
3-(2-Fluoropyridin-4-yl)-2-methyl-6-piperazin-1-ylimidazo[1,2-b]pyridazine;
2,7,8-Trimethyl-6-piperazin-1-yl-3-pyridin-4-ylimidazo[1,2-b]pyridazine and its hydrochloride (3:1);
2-Methoxymethyl-6-piperazin-1-yl-3-pyridin-4-ylimidazo[1,2-b]pyridazine;
2-Ethyl-6-piperazin-1-yl-3-pyridin-4-yl-imidazo[1,2-b]pyridazine and its hydrochloride (3:1);
2-Ethyl-6-piperazin-1-yl-3-(2-methylpyridin-4-yl)imidazo[1,2-b]pyridazine;
2-Isopropyl-6-piperazin-1-yl-3-pyridin-4-ylimidazo[1,2-b]pyridazine;
2-Isopropyl-3-(2-methylpyridin-4-yl)-6-piperazin-1-ylimidazo[1,2-b]pyridazine;
2-Cyclopropyl-6-piperazin-1-yl-3-pyridin-4-ylimidazo[1,2-b]pyridazine;
2-Cyclopropyl-3-(2-methylpyridin-4=y)-6-piperazin-1-ylimidazo[1,2-b]pyridazine;
4-(2-Cyclopropyl-6-piperazin-1-ylimidazo[1,2-b]pyridazin-3-yl)pyridin-2-ylamine;
2-Cyclopropyl-3-(2-methoxypyridin-4-yl)-6-piperazin-1-ylimidazo[1,2-b]pyridazine;
3-(2-Chloropyridin-4-yl)-2-cyclopropyl-6-piperazin-1-ylimidazo[1,2-b]pyridazine;
2-Isobutyl-6-piperazin-1-yl-3-pyridin-4-ylimidazo[1,2-b]pyridazine and its hydrochloride (3:1);
2-Cyclopropylmethyl-6-piperazin-1-yl-3-pyridin-4-ylimidazo[1,2-b]pyridazine;
2-Cyclobutyl-6-piperazin-1-yl-3-pyridin-4-ylimidazo[1,2-b]pyridazine;
(R,S)-2-Methyl-6-(3-methylpiperazin-1-yl)-3-pyridin-4-ylimidazo[1,2-b]pyridazine;
(R,S)-2-Methyl-6-(3-methylpiperazin-1-yl)-3-(2-methylpyridin-4-yl)imidazo[1,2-b]pyridazine;
(R,S)-2-Cyclopropyl-6-(3-methylpiperazin-1-yl)-3-(2-methylpyridin-4-yl)imidazo[1,2-b]-pyridazine;
(R,S)-4-[2-Cyclopropyl-6-(3-methylpiperazin-1-yl)imidazo[1,2-b]pyridazin-3-yl]pyridin-2-ylamine;
(R,S)-2-Cyclopropyl-3-(2-methoxypyridin-4-yl)-6-(3-methylpiperazin-1-yl)imidazo[1,2-b]-pyridazine;
(R,S)-3-(2-Chloropyridin-4-yl)-2-cyclopropyl-6-(3-methylpiperazin-1-yl)imidazo[1,2-b]-pyridazine;
2-Methyl-6-((R)-3-methylpiperazin-1-yl)-3-pyridin-4-ylimidazo[1,2-b]pyridazine;
2-Methyl-6-((R)-3-methylpiperazin-1-yl)-3-(2-methylpyridin-4-yl)imidazo[1,2-b]pyridazine;
4-[2-Methyl-6-((R)-3-methylpiperazin-1-yl)imidazo[1,2-b]pyridazin-3-yl]pyridin-2-ylamine;
Methyl-{4-[2-methyl-6-((R)-3-methylpiperazin-yl)imidazo[1,2-b]pyridazin-3-yl]pyridin-2-yl}-amine;
3-(2-Methoxypyridin-4-yl)-2-methyl-6-((R)-3-methylpiperazin-1-yl)imidazo[1,2-b]pyridazine;
2-Ethyl-6-((R)-3-methylpiperazin-1-yl)-3-pyridin-4-ylimidazo[1,2-b]pyridazine;
2-Ethyl-3-(2-fluoropyridin-4-yl)-6-((R)-3-methylpiperazin-1-yl)imidazo[1,2-b]pyridazine;
2-Cyclopropylmethyl-6-((R)-3-methylpiperazin-1-yl)-3-pyridin-4-ylimidazo[1,2-b]pyridazine;
2-Cyclopropylmethyl-6-((R)-3-methylpiperazin-1-yl)-3-(2-methylpyridin-4-yl)imidazo[1,2-b]-pyridazine;
2-Methyl-6-((S)-3-methylpiperazin-1-yl)-3-pyridin-4-ylimidazo[1,2-b]pyridazine;
3-(2-Methoxypyridin-4-yl)-2-methyl-6-((S)-3-methylpiperazin-1-yl)imidazo[1,2-b]pyridazine;
2-Cyclopropyl-6-((S)-3-methylpiperazin-1-yl)-3-pyridin-4-ylimidazo[1,2-b]pyridazine;
4-[2-Methyl-6-((S)-3-methylpiperazin-1-yl)imidazo[1,2-b]pyridazin-3-yl]pyridin-2-ylamine;
2-Methyl-6-(4-methylpiperazin-1-yl)-3-pyridin-4-ylimidazo[1,2-b]pyridazine;
6-(4-Methylpiperazin-1-yl)-3-pyridin-4-yl-2-trifluoromethylimidazo[1,2-b]pyridazine;
[4-(2-Methyl-3-pyridin-4-ylimidazo[1,2-b]pyridazin-6-yl)piperazin-2-yl]methanol;
6-(4-Ethylpiperazin-1-yl)-2-methyl-3-pyridin-4-ylimidazo[1,2-b]pyridazine and its hydrochloride (3:1);
6-(4-Ethylpiperazin-1-yl)-2-methyl-3-(2-methylpyridin-4-yl)imidazo[1,2-b]pyridazine;
6-(4-Isopropylpiperazin-1-yl)-2-methyl-3-pyridin-4-ylimidazo[1,2-b]pyridazine and its hydrochloride (3:1);
6-(4-Isopropylpiperazin-1-yl)-2-methyl-3-(2-methylpyridin-4-yl)imidazo[1,2-b]pyridazine;
4-[6-(4-Isopropylpiperazin-1-yl)-2-methylimidazo[1,2-b]pyridazin-3-yl]pyridin-2-yl}-amine and its hydrochloride (3:1);
6-(4-Cyclobutylpiperazin-1-yl)-2-methyl-3-pyridin-4-ylimidazo[1,2-b]pyridazine;
4-[6-(4-Cyclobutylpiperazin-1-yl)-2-methylimidazo[1,2-b]pyridazin-3-yl]pyridin-2-ylamine;
6-(3,3-Dimethylpiperazin-1-yl)-2-methyl-3-(2-methylpyridin-4-yl)imidazo[1,2-b]pyridazine;
{4-[6-(3,3-Dimethylpiperazin-1-yl)-2-methylimidazo[1,2-b]pyridazin-3-yl]pyridin-2-yl}methylamine;
2-Cyclopropyl-6-(3,3-dimethylpiperazin-1-yl)-3-(2-methylpyridin-4-yl)imidazo[1,2-b]-pyridazine;
4-[2-Cyclopropyl-6-(3,3-dimethylpiperazin-1-yl)imidazo[1,2-b]pyridazin-3-yl]pyridin-2-ylamine;
2-Cyclopropyl-6-(3,3-dimethylpiperazin-1-yl)-3-(2-fluoropyridin-4-yl)imidazo[1,2-b]pyridazine;
6-(cis-3,5-Dimethylpiperazin-1-yl)-2-methyl-3-pyridin-4-ylimidazo[1,2-b]pyridazine and its hydrochloride (3:1);
6-(cis-3,5-Dimethylpiperazin-1-yl)-2-methyl-3-(2-methylpyridin-4-yl)imidazo[1,2-b]pyridazine;
4-[6-(cis-3,5-Dimethylpiperazin-1-yl)-2-methylimidazo[1,2-b]pyridazin-3-yl]pyridin-2-ylamine;
{4-[6-(cis-3,5-Dimethylpiperazin-1-yl)-2-methylimidazo[1,2-b]pyridazin-3-yl]pyridin-2-yl}-methylamine;
2-Cyclopropyl-6-(cis-3,5-dimethylpiperazin-1-yl)-3-pyridin-4-ylimidazo[1,2-b]pyridazine;
4-[2-Cyclopropyl-6-(cis-3,5-dimethylpiperazin-1-yl)imidazo[1,2-b]pyridazin-3-yl]pyridin-2-ylamine;
6-(S)-Hexahydropyrrolo[1,2-a]pyrazin-2-yl-2-methyl-3-pyridin-4-ylimidazo[1,2-b]pyridazine;
6-(S)-Hexahydropyrrolo[1,2-a]pyrazin-2-yl-2-methyl-3-(2-methylpyridin-4-yl)imidazo[1,2-b]-pyridazine;
6-(1S,4S)-2,5-Diazabicyclo[2.2.1]hept-2-yl-2-methyl-3-pyridin-4-ylimidazo[1,2-b]pyridazine;
(R,S)-6-(2,5-Diazabicyclo[2.2.1]hept-2-yl)-2-methyl-3-(2-methylpyridin-4-yl)imidazo[1,2-b]-pyridazine and its hydrobromide (1:1);
4-[2-Cyclopropyl-6-(1,4-diazabicyclo[3.2.2]non-4-yl)-3-(2-methylpyridin-4-yl)imidazo[1,2-b]-pyridazine;
(R,S)-6-(Hexahydropyrrolo[3,4-b]pyrrol-5(1H)-yl)-2-methyl-3-(2-methylpyridin-4-yl)imidazo[1,2-b]pyridazine;
6-Hexahydropyrrolo[3,4-c]pyrrol-2(1H)-yl-2-methyl-3-pyridin-4-ylimidazo[1,2-b]pyridazine;
6-Hexahydropyrrolo[3,4-c]pyrrol-2(1H)-yl-2-methyl-3-(2-methylpyridin-4-yl)-imidazo[1,2-b]-pyridazine and its hydrochloride (3:1);
4-(6-Hexahydropyrrolo[3,4-c]pyrrol-2(1H)-yl-2-methylimidazo[1,2-b]pyridazin-3-yl)pyridin-2-ylamine;
[4-(6-Hexahydropyrrolo[3,4-c]pyrrol-2(1H)-yl-2-methylimidazo[1,2-b]pyridazin-3-yl)pyridin-2-yl]-methylamine;
2-Methyl-6-(5-methylhexahydropyrrolo[3,4-c]pyrrol-2(1H)-yl)-3=pyridin-4-ylimidazo[1,2-b]-pyridazine;
2,7-Dimethyl-6-(5-methylhexahydropyrrolo[3,4-c]pyrrol-2(H)-yl)-3-pyridin-4-yl-imidazo[1,2-b]pyridazine;
6-(−5-Isopropylhexahydropyrrolo[3,4-c]pyrrol-2(1H)-yl)-2-methyl-3-pyridin-4-ylimidazo[1,2-b]-pyridazine;
4-[6-(−5-Isopropylhexahydropyrrolo[3,4-c]pyrrol-2(1H)-yl)-2-methylimidazo[1,2-b]pyridazin-3-yl]pyridin-2-ylamine;
6-Hexahydropyrrolo[3,4-c]pyrrol-2(1H)-yl-3-(2-methoxypyridin-4-yl)-2-methylimidazo[1,2-b]-pyridazine;
(R,S)-2-Methyl-6-(octahydropyrrolo[3,4-b]pyridin-6-yl)-3-pyridin-4-ylimidazo[1,2-b]-pyridazine;
6-(2,7-Diazaspiro[3.5]non-7-yl)-2-methyl-3-(2-methylpyridin-4-yl)imidazo[1,2-b]pyridazine;
(R,S)-6-(2,7-Diazaspiro[4.5]dec-2-yl)-2-methyl-3-pyridin-4-ylimidazo[1,2-b]pyridazine;
9-(2-Methyl-3-pyridin-4-yl-imidazo[1,2-b]pyridazin-6-yl)-2,9-diazaspiro[5.5]undecane and its hydrochloride (3:1);
9-[2-Methyl-3-(2-methylpyridin-4-yl)imidazo[1,2-b]pyridazin-6-yl]-2,9-diazaspiro[5.5]-undecane and its hydrochloride (3:1);
9-(2-Methyl-3-pyridin-4-ylimidazo[1,2-b]pyridazin-6-yl)-1-oxa-4,9-diazaspiro[5.5]undecane and its hydrochloride (3:1);
4-[2-Methyl-6-(1-oxa-4,9-diazaspiro[5.5]undec-9-yl)imidazo[1,2-b]pyridazin-3-yl]pyridin-2-ylamine and its hydrochloride (3:1);
2-Methyl-3-pyridin-4-yl-6-(4-pyrrolidin-1-ylpiperidin-1-yl)imidazo[1,2-b]pyridazine;
2-Methyl-3-(2-methylpyridin-4-yl)-6-(4-pyrrolidin-1-ylpiperidin-1-yl)imidazo[1,2-b]pyridazine;
4-[2-Methyl-6-(4-pyrrolidin-1-ylpiperidin-1-yl)imidazo[1,2-b]pyridazin-3-yl]pyridin-2-ylamine;
(R,S)-6-[1,3′]Bipyrrolidinyl-1′-yl-2-methyl-3-pyridin-4-ylimidazo[1,2-b]pyridazine.

The invention also provides a process for preparing compounds of the invention of formula (I).

In accordance with the invention the compounds of general formula (I) may be prepared according to the general process which is described in scheme 1 below.

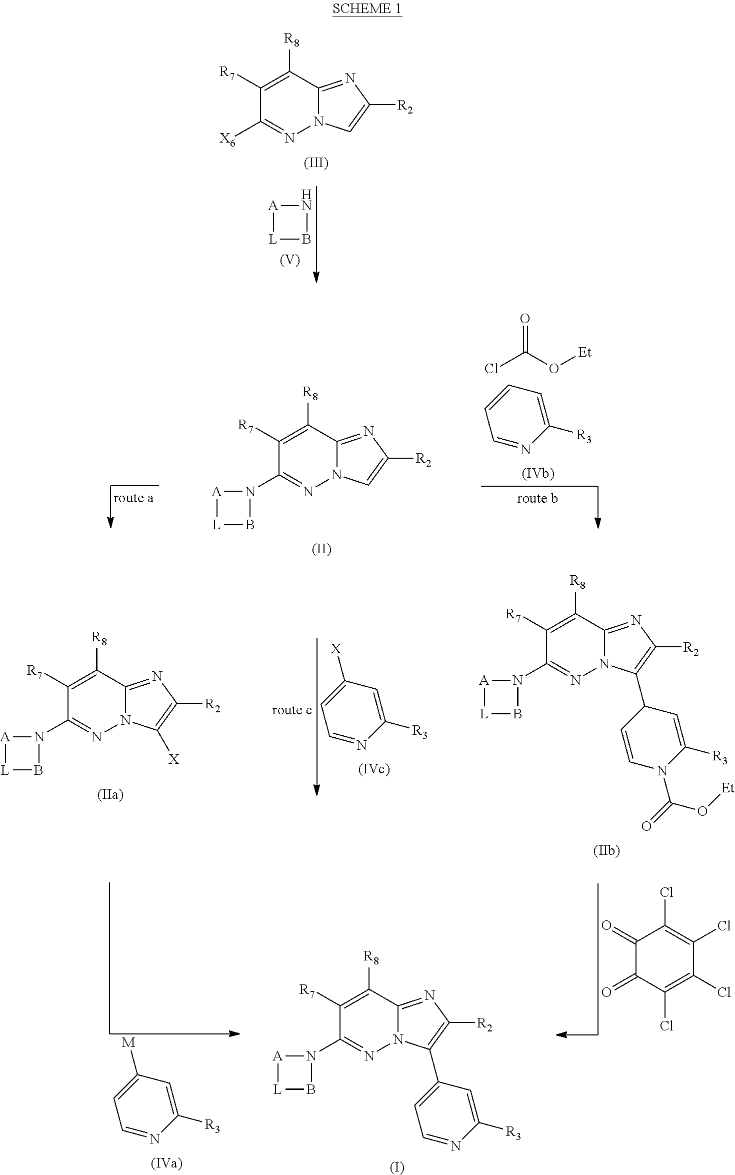

In the text below, a leaving group is a group which is readily cleavable from a molecule by breaking a heterolytic bond, with the departure of an electron pair. This group may thus be replaced easily by another group in the course of a substitution reaction, for example. Such leaving groups are, for example, halogens or an activated hydroxyl group such as a mesyl, tosyl, triflate, acetyl, etc. Examples of leaving groups and also references for their preparations are given in Advances in Organic Chemistry, J. March, 3^{rd} Edition, Wiley Interscience, pp. 310-316.

In the text below, a protective group is a group which allows a reactive function such as a hydroxyl or an amine to be masked during a synthesis, and that allows the reactive function to be restored intact at the end of synthesis, after a step referred to as deprotection. Examples of protective groups and also of methods of protection and deprotection are given in Protective Groups in Organic Synthesis, Greene et al., 2^{nd} Edition (John Wiley & Sons, Inc., New York), 1991.

### Scheme 1: Preliminary Introduction of the Amine

### Route a: Introduction of the Pyridine—Stille or Suzuki Coupling

In general and as illustrated in scheme 1, the 2-alkyl-6-cycloamino-3-pyridin-4-ylimidazo-[1,2-b]pyridazine derivatives of general formula (I) in which R₂, R₃, A, L, B, R₇ and R₈ are as defined above may be prepared in two steps, starting from a 2-alkyl-6-cycloaminoimidazo[1,2-b]pyridazine derivative of general formula (II) in which R₂, A, L, B, R₇ and R₈ are as defined above.

The derivative (II) is selectively brominated or iodinated in position 3 by treatment using N-bromo- or iodosuccinimide or iodine monochloride in a polar solvent such as acetonitrile, tetrahydrofuran, methanol or chloroform, to give the 6-amino-3-iodo- or -3-bromoimidazo-[1,2-b]pyridazine derivative of general formula (IIa) in which R₂, A, L, B, R₇ and R₈ are as defined above and X represents a bromine or iodine atom. This derivative is converted to a compound of the invention of general formula (I) by coupling under Stille or Suzuki conditions with a stannane or a pyridine boronate of general formula (IVa) in which R₃ is as defined above and M represents a trialkylstannyl group, usually a tributylstannyl group, or a dihydroxyboryl or dialkyloxyboryl group, usually a 4,4,5,5-tetramethyl-1,3,3,2-dioxaborolan-2-yl group.

The couplings according to the Stille method are performed, for example, by heating in the presence of a catalyst such as tetrakis(triphenylphosphine)palladium or copper iodide in a solvent such as N,N-dimethylacetamide.

The couplings according to the Suzuki method are performed, for example by heating in the presence of a catalyst such as 1,1′-bis(diphenylphosphino)ferrocenedichloropalladium and an inorganic base such as caesium carbonate in a solvent mixture such as tetrahydrofuran and water.

### Route b: Introduction of the Pyridine—Nucleophilic Addition to Pyridinium

Alternatively the 2-alkyl-6-cycloamino-3-pyridin-4-ylimidazo[1,2-b]pyridazine derivatives of general formula (I) in which R₂, R₃, A, L, B, R₇ and R₈ are as defined above may also be prepared in two steps, starting from a 2-alkyl-6-cycloaminoimidazo[1,2-b]pyridazine derivative of general formula (II) in which R₂, A, L, B, R₇ and R₈ are as defined above.

The reaction of a 2-alkyl-6-cycloaminoimidazo[1,2-b]pyridazine derivative of general formula (II) with a mixture of a pyridine derivative of general formula (IVb) in which R₃ represents a hydrogen atom or a C₁₋₃-alkyl group and alkyl chloroformate, for example ethyl chloroformate, leads to the derivative of general formula (IIb) in which R₂, A, L, B, R₇ and R₈ are as defined above and in which R₃ represents a hydrogen atom or a C₁₋₃-alkyl group. The derivative of general formula (IIb) is then oxidized using ortho-chloranil in a solvent such as toluene, to give the derivatives of the invention of general formula (I) in which R₂, A, L, B, R₇ and R₈ are as defined above and in which R₃ represents a hydrogen atom or a C₁₋₃-alkyl group.

### Route c: INTRODUCTION of the Pyridine—Metal-Catalysed C—H Arylation

Lastly, the 2-alkyl-6-cycloamino-3-pyridin-4-ylimidazo[1,2-b]pyridazine derivatives of general formula (I) in which R₂, R₃, R₇, R₈, A, L, and B are as defined above may be prepared directly from a 2-alkyl-6-cycloaminoimidazo[1,2-b]pyridazine derivative of general formula (II), in which R₂, R₇, R₈, A, L and B are as defined above by metal-catalysed coupling with a pyridine derivative of general formula (IVc) in which R₃ is as defined above and X represents a halogen atom, more particularly iodine. This coupling may be carried out in the presence of a catalyst such as palladium acetate and an inorganic base such as potassium carbonate and in an aprotic polar solvent such as dimethylformamide.

The 2-alkyl-6-cycloaminoimidazo[1,2-b]pyridazine derivatives of general formula (II) in which R₂, A, L, B, R₇ and R₈ are as defined above may be prepared from a 2-alkyl-6-cycloaminoimidazo[1,2-b]pyridazine derivative of general formula (III), in which R₂, R₇ and R₈ are as defined above and X₆ represents a leaving group such as a halogen, by treatment using an amine of general formula (V) in which A, L and B are as defined above. This reaction may be performed by heating the reactants in a polar solvent such as dimethyl sulphoxide or aliphatic alcohols, for example pentanol.

Scheme 2: Preliminary Functionalization of Position 3 of the imidazo[1,2-b]pyridazine

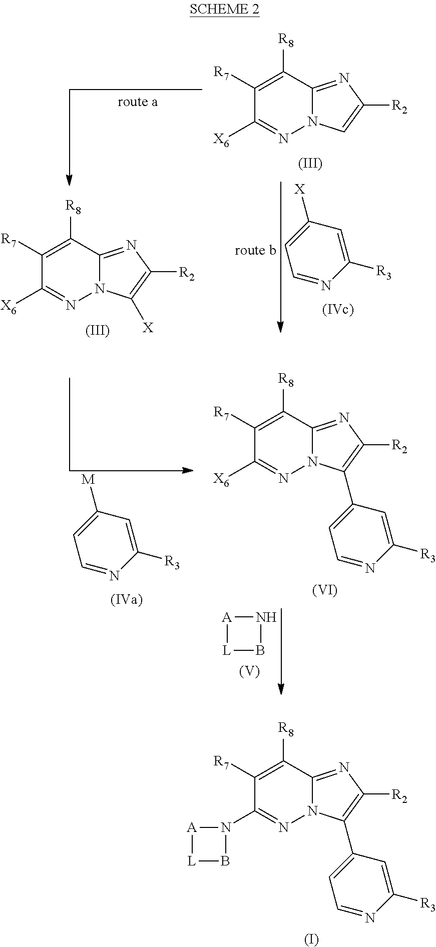

In general and as illustrated in scheme 2, the 2-alkyl-6-cycloamino-3-pyridin-4-yl-imidazo-[1,2-b]pyridazine derivatives of general formula (I) in which R₂, R₃, A, L, B, R₇ and R₈ are as defined above may be prepared from a 2-alkyl-3-pyridin-4-ylimidazo[1,2-b]pyridazine derivative of general formula (VI), in which R₂, R₃, R₇ and R₈ are as defined above and X₆ represents a leaving group such as a halogen, by treatment using an amine of general formula (V) in which A, L and B are as defined above. This reaction may be performed by heating of the reactants in a polar solvent such as dimethyl sulphoxide or aliphatic alcohols, for example pentanol.

### Route a: Introduction of the Pyridine—Stille or Suzuki Coupling

The 2-alkyl-3-pyridin-4-ylimidazo[1,2-b]pyridazine derivatives of general formula (VI) as defined above may be prepared in two steps from a 2-alkyl-imidazo[1,2-b]pyridazine derivative of general formula (III) as defined above:

The derivative (III) is brominated or iodinated selectively in position 3 by treatment using N-bromo- or iodosuccinimide or iodine monochloride in a polar solvent such as acetonitrile, tetrahydrofuran, methanol or chloroform, to give the 2-alkyl-3-iodo- or 3-bromo-imidazo-[1,2-b]pyridazine derivative of general formula (IIIa), in which R₂, R₇ and R₈ are as defined above and X₆ represents a leaving group such as a halogen. This derivative is converted to derivative (VI) by coupling as defined above under Stille or Suzuki conditions with a stannane or a pyridine boronate of general formula (IVa) in which R₃ is as defined above and M represents a trialkylstannyl group, usually a tributylstannyl group, or a dihydroxyboryl or dialkyloxyboryl group, usually a 4,4,5,5-tetramethyl-1,3,3,2-dioxaborolan-2-yl group.

The couplings according to the Stille method are performed, for example, by heating in the presence of a catalyst such as tetrakis(triphenylphosphine)palladium or copper iodide in a solvent such as N,N-dimethylacetamide.

The couplings according to the Suzuki method are performed, for example by heating in the presence of a catalyst such as 1,1′-bis(diphenylphosphino)ferrocenedichloropalladium and an inorganic base such as caesium carbonate in a solvent mixture such as tetrahydrofuran and water.

### Route b: Introduction of the Pyridine—Metal-Catalysed C—H Arylation

Alternatively, the 2-alkyl-3-pyridin-4-ylimidazo[1,2-b]pyridazine derivatives of general formula (VI) as defined above may be prepared directly in one step from a 2-alkyl-imidazo-[1,2-b]pyridazine derivative of general formula (III) as defined above by metal-catalysed coupling with a pyridine derivative of general formula (IVc) in which R₃ is as defined above and X represents a halogen atom, more particularly iodine. This coupling may be carried out in the presence of a catalyst such as palladium acetate and an inorganic base such as potassium carbonate and in an aprotic polar solvent such as dimethylformamide.

In the specific case of compounds of general structure (I) or (VI) for which R₃ represents a group —NR₄R₅, and the group R₅ represents a C₁₋₄-alkyl, C₃₋₇-cycloalkyl or C₃₋₇-cycloalkyl-C₁₋₆-alkyl group, these compounds may be prepared by alkylation of the corresponding precursor in which the group R₅ represents a hydrogen, using an alkyl halide of formula R₅—X for which X represents a leaving group as defined above. This reaction may be carried out by heating in the presence of a base such as sodium hydride and in a solvent such as dimethylformamide or tetrahydrofuran. In the very specific case in which the groups R₄ and R₅ each represent a hydrogen, a monoalkylation may be carried out by using a protective group such as a tert-butyloxycarbonyl for temporary masking of one of the two hydrogens.

### Synthesis of Precursors

The 2-alkyl-imidazo[1,2-b]pyridazine derivatives of general formula (III) in which R₂, R₇ and R₈ are as defined above and X₆ represents a leaving group are known or may be prepared by analogy with methods described in the literature (Abignente, Enrico; Caprariis, Paolo de; Patscot, Rosaria; Sacchi, Antonella; J. Heterocycl. Chem.; 23; 1986; 1031-1034; Barlin, Gordon B.; Davies, Les P.; Ireland, Stephen J.; Ngu, Maria M. L.; Zhang, Jiankuo; Aust. J. Chem.; EN; 45; 4; 1992; 731-749; Mourad, Alaa E.; Wise, Dean S.; Townsend, Leroy B.; J. Heterocycl. Chem.; 30; 5; 1993; 1365-1372; Pollak et al.; Tetrahedron; 24; 1968; 2623; Hervet, Maud; Galtier, Christophe; Enguehard, Cecile; Gueiffier, Alain; Debouzy, Jean-Claude; Journal of Heterocyclic Chemistry (2002), 39(4), 737-742).

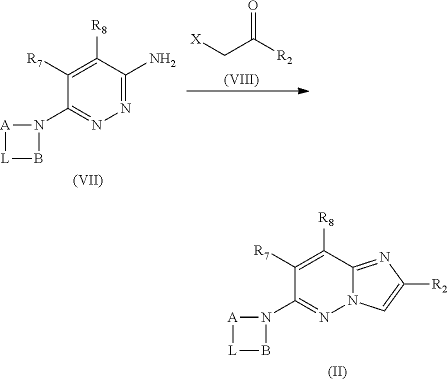

The 2-alkyl-6-cycloamino-3-pyridin-4-ylimidazo[1,2-b]pyridazine derivatives of general formula (II) in which R₂, A, L, B, R₇ and R₈ are as defined above may be prepared by analogy with methods that are described in the literature (for example, Watanabe et al.; Synthesis; 1977; 761; Jurgee et al.; J. Heterocycl. Chem.; 12; 1975; 253,255.; Werbel, L. M.; Zamora, M. L.; J. Heterocycl. Chem.; 2; 1965; 287-290; Yoneda et al.; Chem. Pharm. Bull.; 12; 1964; 1351, 1353, 1354; Tomoyasu; Iizawa, Yuji; Okonogi, Kenji; Miyake, Akio; J. Antibiot.; 53; 10; 2000; 1053-1070).

They are commonly prepared by condensing a pyridazin-3-ylamine derivative of general formula (VII) in which A, L, B, R₇ and R₈ are as defined above with a 2-bromo-, chloro- or iodoethan-1-one derivative of general formula (VIII) in which R₂ is as defined above.

The reaction may be performed by heating the reactants in a polar solvent such as ethanol or butanol.

In the synthesis schemes above, the starting compounds and the reactants, when their method of preparation is not described, are available commercially or described in the literature, or else may be prepared by methods which are described therein or which are known to a person skilled in the art.

### Protective Groups

For the compounds of general formula (I), (II), (IIa) or (IIc) as defined above, and in the case where the group N-A-L-B includes a primary or secondary amine function, this function may optionally be protected during the synthesis by protective groups that are known to a person skilled in the art, for example a benzyl or a tert-butyloxycarbonyl.

For the compounds of general formula (I) or (VI) as defined above and in the case where the group R₃ includes a primary or secondary amine function, this function may optionally be protected by protective groups that are known to a person skilled in the art, for example a benzyl or a t-butyloxycarbonyl.

The products of general structure (I) as defined above are obtained according to the processes described after an additional final step of deprotection of the protective group in accordance with the customary conditions that are known to a person skilled in the art.

The examples that follow describe the preparation of certain compounds in accordance with the invention. These examples are not limitative and serve only to illustrate the present invention. The numbers of the compounds exemplified relate to those which are given in the table below, which illustrates the chemical structures and physical properties of some compounds according to the invention.

### EXAMPLE 1

### Compound 39

### 2-Methyl-6-(4-methylpiperazin-1-yl)-3-pyridin-4-ylimidazo[1,2-b]pyridazine

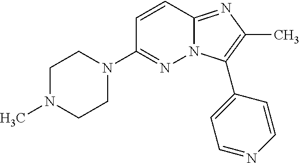

Step 1.1.2-Methyl-6-(4-methylpiperazin-1-yl)-imidazo[1,2-b]pyridazine

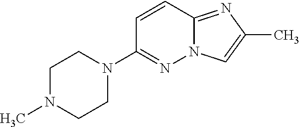

A solution of 4.40 g (26.3 mmol) of 6-chloro-2-methylimidazo[1,2-b]pyridazine (CAS 14793-00-1; Mourad, Alaa E.; Wise, Dean S.; Townsend, Leroy B.; J. Heterocycl. Chem.; 30; 5; 1993; 1365-1372) in 58 ml of 1-methylpiperazine is heated at reflux for 24 hours. The mixture is then poured into water and the product is extracted with dichloromethane. The organic phases are combined, dried over sodium sulphate and concentrated under reduced pressure. The resulting brown oil is purified by chromatography on silica gel, eluting with a mixture of dichloromethane, methanol and aqueous ammonia (95/5/0,5), to give 4.79 g of beige powder following recrystallization from diisopropyl ether and drying.

m.p.: 108-110° C.

¹H NMR (CDCl₃) δ: 7.65 (d, 1H), 7.50 (s, 1H), 6.80 (d, 1H), 3.55 (m, 4H), 2.60 (m, 4H), 2.45 (s, 3H), 2.40 (s, 3H) ppm.

Step 1.2. 3-Iodo-2-methyl-6-(4-methylpiperazin-1-yl)imidazo[1,2-b]pyridazine

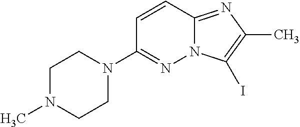

A solution of 5.50 g (23.8 mmol) of 2-methyl-6-(4-methylpiperazin-1-yl)-imidazo[1,2-b]-pyridazine in 100 ml of dichloromethane is admixed with 5.89 g (26.2 mmol) of N-iodosuccinimide. The mixture is stirred for an hour and a half at ambient temperature and then the solvent is evaporated under reduced pressure. The resulting solid residue is taken up with 5% aqueous sodium thiosulphate solution and the product is extracted with dichloromethane. The organic phases are combined, dried over sodium sulphate and concentrated under reduced pressure, to give 6.37 g of beige powder following recrystallization from acetonitrile, rinsing with diethyl ether and drying.

m.p.: 136-138° C.

¹H NMR (CDCl₃) δ: 7.55 (d, 1H), 6.80 (d, 1H), 3.60 (m, 4H), 2.60 (m, 4H), 2.50 (s, 3H), 2.40 (s, 3H) ppm.

Step 1.3. 2-Methyl-6-(4-methylpiperazin-1-yl)-3-pyridin-4-ylimidazo[1,2-b]pyridazine

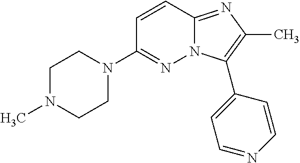

A mixture of 0.50 g (1.40 mmol) of 3-iodo-2-methyl-6-(4-methylpiperazin-1-yl)imidazo[1,2-b]-pyridazine, 0.207 g (1.68 mmol) of (pyridin-4-yl)boronic acid, 0.30 g (2.8 mmol) of sodium carbonate and 46 mg (0.06 mmol) of a complex of 1,1′-bis(diphenylphosphino)ferrocenedichloropalladium(II) and dichloromethane (PdCl₂ (dppf).CH₂Cl₂) in 10 ml of a mixture of dimethoxyethane and water (7/3) is admixed with 3.0 g (9.3 mmol) of caesium carbonate and the mixture is heated at reflux under argon for 18 hours. Then 1.72 g (1.40 mmol) of (pyridin-4-yl)boronic acid and 46 mg (0.06 mmol) of the complex of 1,1′-bis(diphenylphosphino)ferrocenedichloropalladium(II) and dichloromethane are added. The reaction is continued for 3 more hours at reflux and then the mixture is poured into 200 ml of water. The product is then extracted with dichloromethane, the organic phase is dried over sodium sulphate and filtered and the solvent is evaporated under reduced pressure, to give a brown solid. The product is purified by chromatography on silica gel, eluting with a mixture of dichloromethane, methanol and aqueous ammonia (95/5/0.5), to give 0.308 g of beige powder following recrystallization from acetonitrile, rinsing with diisopropyl ether and drying.

m.p.: 150-152° C.

¹H NMR (CDCl₃) δ: 8.70 (d, 2H), 7.7 (m, 3H), 6.90 (d, 1H), 3.55 (m, 4H), 2.65 (s, 3H), 2.60 (m, 4H), 2.40 (s, 3H) ppm.

Step 1.4. Alternative strategy: 2-Methyl-6-(4-methylpiperazin-1-yl)-3-pyridin-4-ylimidazo-[1,2-b]pyridazine

A mixture of 1.00 g (4.32 mmol) of 2-methyl-6-(4-methylpiperazin-1-yl)imidazo[1,2-b]-pyridazine, 1.06 g (5.19 mmol) of 4-iodopyridine, 0.717 g (5.19 mmol) of sodium carbonate and 49 mg of palladium acetate in 10 ml of dimethylformamide is heated at 140° C. for 18 hours. After cooling, the mixture is poured into water. Ethyl acetate is added and the mixture is filtered on a Buchner funnel. The organic phase is separated, washed with water and dried over sodium sulphate. The solvent is removed under reduced pressure and the residue is purified by chromatography on a silica gel column, eluting with a mixture of dichloromethane, methanol and aqueous ammonia (98/2/0.2), to give 0.7 g of product after drying.

m.p.: 154-156° C.

### EXAMPLE 2

### Compound 19

### 2-Cyclobutyl-6-piperazin-1-yl-3-pyridin-4-ylimiidazo[1,2-b]-pyridazine

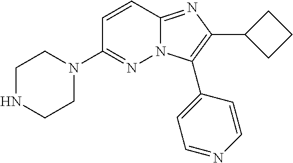

Step 2.1. 6-(4-Benzylpiperazin-1-yl)pyridazin-3-ylamine

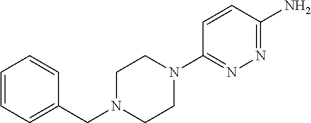

48.9 g (278 mmol) of 1-benzylpiperazine and 12.0 g (92.6 mmol) of 3-amino-6-chloropyridazine are heated at 160° C. for 1 hour. The resulting brown oil is poured into 500 ml of aqueous sodium bicarbonate solution and the product is extracted with dichloromethane. The organic phase is dried and then concentrated under reduced pressure. The resulting oil is triturated in diethyl ether and 20.5 g of solid are isolated after filtration and drying.

Yield: 82%

¹H NMR (CDCl₃) δ: 7.45-7.65 (m, 6H), 7.20 (s, 2H), 5.5 (broad unresolved peak, 2H) 3.80 (s, 2H), 3.60-3.75 (m, 4H), 2.80-2.85 (m, 4H) ppm.

Step 2.2. 6-(4-Benzylpiperazin-1-yl)-2-cyclobutylimidazo[1,2-b]pyridazine

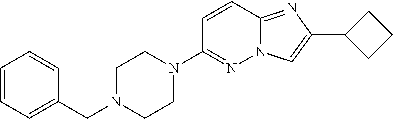

A solution of 2.0 g (7.4 mol) of 6-(4-benzylpiperazin-1-yl)pyridazin-3-ylamine, obtained in step 2.1., and 1.6 g (8.9 mmol) of 2-bromo-1-cyclobutylethanone (CAS: 128312-69-6) in ml of n-butanol is heated at 90° C. for 1 hour 40 minutes. The mixture is then poured into 250 ml of aqueous sodium hydrogen carbonate solution and the product is extracted with ethyl acetate. The organic phase is dried over sodium sulphate and filtered and the solvent is evaporated under reduced pressure. The resulting brown oil is purified by chromatography on 90 g of silica gel, eluting with a mixture of dichloromethane, methanol and aqueous ammonia (97/3/0.3), to give 1.6 g of yellow oil.

Yield: 62%

¹H NMR (CDCl₃) δ: 7.65 (d, 1H), 7.50 (s, 1H), 7.20-7.40 (m, 5H), 6.75 (d, 1H), 3.75 (m, 1H), 3.60 (s, 2H), 3.45-3.55 (m, 4H), 2.50-2.65 (m, 4H), 1.8-2.5 (m, 6H) ppm.

Step 2.3. Ethyl 4-[6-(4-benzylpiperazin-1-yl)-2-cyclobutylimidazo[1,2-b]pyridazin-3-yl]-4H-pyridine-1-carboxylate

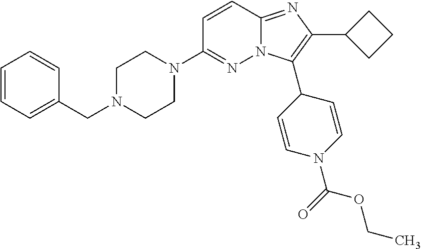

A solution of 1.6 g (4.6 mmol) of 6-(4-benzylpiperazin-1-yl)-2-cyclobutylimidazo[1,2-b]-pyridazine, obtained in step 2.2., in 15 ml of pyridine, cooled to 0° C., is admixed dropwise with 8.8 ml (92 mmol) of ethyl chloroformate, the temperature being held at 00° C. The mixture is then allowed to return to ambient temperature, during which precipitate formed disappears.

The mixture is cooled again to 0° C. and 8.8 ml (92 mmol) of ethyl chloroformate are added dropwise, the temperature being held at 0° C. Again the mixture is allowed to return to ambient temperature, during which precipitate formed disappears.

The mixture is then poured into 300 ml of water and the product is extracted with ethyl acetate. The organic phase is washed with water, dried over sodium sulphate and then concentrated under reduced pressure. The residue is subsequently coevaporated a number of times with toluene, after which the product is crystallized and eventually recrystallized from acetonitrile.

This gives 1.0 g of white powder after filtration and drying.

m.p.: 140-142° C.

Yield: 45%

¹H NMR (CDCl₃) δ: 7.60 (d, 1H), 7.10-7.30 (m, 5H), 6.80-7.00 (m, 2H), 6.65 (d, 1H), 4.60-4.9 (m, 3H), 4.00-4.25 (m, 2H), 3.7 (m, 1H), 3.50 (s, 2H), 3.25-3.40 (m, 4H), 2.05-2.50 (m, 8H), 1.75-2.05 (m, 2H), 1.20 (t, 3H) ppm.

Step 2.4. 6-(4-Benzylpiperazin-1-yl)-2-cyclobutyl-3-pyridin-4-ylimidazo[1,2-b]pyridazine

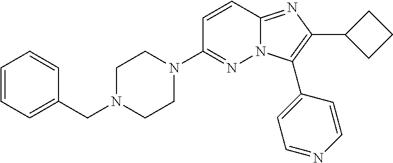

A solution of 1.0 g (2.1 mmol) of ethyl 4-[6-(4-benzylpiperazin-1-yl)-2-cyclobutylimidazo-[1,2-b]pyridazin-3-yl]-4H-pyridine-1-carboxylate, obtained in step 2.3., in 8 ml of toluene is admixed dropwise with 0.55 g (2.25 mmol) of ortho-chloranil in solution in 4 ml of toluene (red solution). After the end of the addition, the mixture is left with stirring at ambient temperature for 30 minutes and then poured into 2N aqueous sodium hydroxide solution. The product is extracted with ethyl acetate and the organic phase is washed with water, dried over sodium sulphate and then concentrated under reduced pressure. The resulting black oil is purified by chromatography on 50 g of silica gel, eluting with a mixture of dichloromethane, methanol and aqueous ammonia (95/5/0.5), to give 0.87 g of yellow oil.

Yield: 100%

¹H NMR (CDCl₃) δ: 8.70 (m, 2H), 7.85 (d, 1H), 7.70 (m, 2H), 7.25-7.45 (m, 5H), 6.90 (d, 1H), 3.90 (q, 1H), 3.60 (s, 2H), 3.55 (m, 4H), 2.50-2.75 (m, 6H), 2.30-2.50 (m, 2H), 1.95-2.20 (m, 2H) ppm.

Step 2.5. 2-Cyclobutyl-6-piperazin-1-yl-3-pyridin-4-ylimidazo[1,2-b]pyridazine

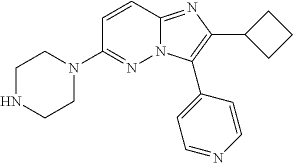

A solution of 0.86 g (2.0 mmol) of 6-(4-benzylpiperazin-1-yl)-2-cyclobutyl-3-pyridin-4-ylimidazo[1,2-b]pyridazine, obtained in step 2.4., in 20 ml of methanol is admixed with 1.9 g of ammonium formate and 0.7 g of palladium on carbon (10%) with a moisture content of 50%. The mixture is heated at reflux for 1 hour and then the catalyst is removed by filtration on a Büchner funnel and rinsing with methanol. The solvent is removed by evaporation and the residue obtained is taken up with dichloromethane and washed with water. The organic phase is washed with water, dried over sodium sulphate and then concentrated under reduced pressure to give a slightly brown-yellow solid.

Recrystallization from acetonitrile, filtration and drying gives 0.33 g of white powder.

m.p.: 189-191° C.

¹H NMR (CDCl₃) δ: 8.70 (pseudo dd, 2H), 7.80 (d, 1H), 7.65 (pseudo dd, 2H), 6.85 (d, 1H), 3.85 (m, 1H), 3.50 (m, 4H), 3.05 (m, 4H), 2.45-2.75 (m, 2H), 2.25-2.45 (m, 2H), 1.85-2.25 (m, 2H) ppm.

### EXAMPLE 3

### Compound 49

### 6-(3,3-Dimethylpiperazin-1-yl)-2-methyl-3-(2-methylpyridin-4-yl)imidazo[1,2-b]pyridazine

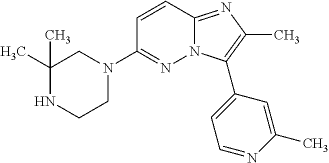

Step 3.1. 6-Chloro-3-iodo-2-methylimidazo[1,2-b]pyridazine

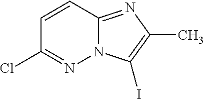

A solution of 7.00 g (41.8 mmol) of 6-chloro-2-methylimidazo[1,2-b]pyridazine (CAS 14793-00-1) in 300 ml of chloroform, cooled at 00° C., is admixed with 10.2 g (62.7 mmol) of iodine monochloride in solution in 20 ml of methanol. The reaction is then left at ambient temperature for 16 hours and subsequently poured into a mixture of an aqueous 5% sodium thiosulphate and sodium hydrogen carbonate solution. The product is extracted with dichloromethane, the organic phase is dried over sodium sulphate and the solvent is evaporated under reduced pressure.

The solid residue is triturated with acetonitrile and then isolated by filtration, to give 8.5 g of a yellow solid after drying.

¹H NMR (CDCl₃) δ: 7.80 (d, 1H), 7.10 (d, 1H), 2.55 (s, 3H) ppm.

Step 3.2. 6-Chloro-2-methyl-3-(2-methylpyridin-4-yl)imidazo[1,2-b]pyridazine

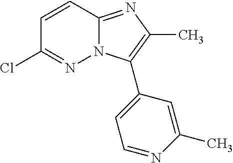

7.70 g (26.2 mmol) of 6-chloro-3-iodo-2-methylimidazo[1,2-b]pyridazine, 4.31 g (31.5 mmol) of 2-methylpyridin-4-ylboronic acid, obtained in step 3.2., 25.6 g (78.7 mmol) of caesium carbonate and 1.93 g (2.36 mmol) of a complex of 1,1′-bis(diphenylphosphino)ferrocenedichloropalladium(II) and dichloromethane (PdC₂ (dppf).CH₂Cl₂) in 480 ml of a mixture of tetrahydrofuran and water (90/10) are heated for 18 hours at reflux. The mixture is poured into water and the product is extracted with ethyl acetate. The organic phase is dried over sodium sulphate and the solvent is evaporated under reduced pressure. The residue is purified on 220 g of silica gel, eluting with a mixture of dichloromethane, methanol and aqueous ammonia (98/2/0.2), to give 5.0 g of a yellow solid. This solid is taken up with a mixture of aqueous hydrochloric acid and ethyl acetate. The aqueous phase is separated off and neutralized with sodium hydrogen carbonate and the product is extracted using chloroform. The organic phase is dried over sodium sulphate and the solvent is evaporated under reduced pressure, to give 4.6 g of a white solid.

¹H NMR (CDCl₃) δ: 8.80 (d, 1H), 7.90 (d, 1H), 7.55 (s, 1H), 7.50 (d, 1H), 7.15 (d, 1H), 2.80 (s, 3H), 2.75 (s, 3H) ppm.

Step 3.3. 6-(3,3-dimethylpiperazin-1-yl)-2-methyl-3-(2-methylpyridin-4-yl)imidazo[1,2-b]-pyridazine

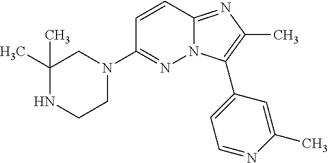

A solution of 0.30 g (1.2 mmol) of 6-chloro-2-methyl-3-(2-methylpyridin-4-yl)imidazo[1,2-b]-pyridazine, obtained in step 3.2., and 0.40 g (3.5 mmol) of 2,2-dimethylpiperazine in 5 ml of pentanol is heated at reflux for 48 hours. The pentanol is then partially evaporated under reduced pressure, and the mixture is taken up with an aqueous solution of hydrochloric acid. This aqueous phase is washed with ethyl acetate and then basified using aqueous sodium hydroxide solution, and the product is extracted with dichloromethane. The aqueous phase is dried over sodium sulphate and the solvent is stripped off under reduced pressure. The residue is purified on 15 g of silica gel, eluting with a mixture of dichloromethane, methanol and aqueous ammonia (94/4/0.4), to give 0.12 g of a white solid.

m.p.: 110-112° C.

¹H NMR (CDCl₃) δ: 8.60 (d, 1H), 7.70 (d, 1H), 7.65 (s, 1H), 7.55 (d, 1H), 6.80 (d, 1H), 3.45 (m, 2H), 3.25 (s, 2H), 3.10 (m, 2H), 2.65 (s, 3H), 2.60 (s, 3H), 1.2 (s, 6H) ppm.

### EXAMPLE 4

### Compound 30

### (−)-3-(2-Methoxypyridin-4-yl)-2-methyl-6-((R)-3-methylpiperazin-1-yl)imidazo[1,2-b]pyridazine

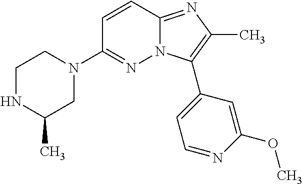

Step 4.1. 6-Chloro-2-methyl-3-(2-methoxypyridin-4-yl)imidazo[1,2-b]pyridazine

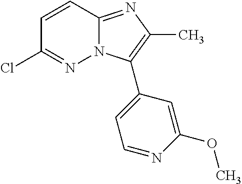

A mixture of 1.15 g (3.92 mmol) of 6-chloro-3-iodo-2-methylimidazo[1,2-b]pyridazine, 0.72 g (4.7 mmol) of 2-methoxypyridin-4-ylboronic acid and 3.8 g (12 mmol) of caesium carbonate in 75 ml of a mixture of tetrahydrofuran and water (90/10) is purged with argon and then 0.29 g (0.35 mmol) of a complex of 1,1′-bis(diphenylphosphino)ferrocenedichloropalladium(II) and dichloromethane (PdCl2(dppf).CH₂Cl₂) is added. After 16 hours of heating at reflux, the mixture is poured into 1N aqueous hydrochloric acid solution which is ice-cold, and the aqueous phase is washed with ethyl acetate and then basified by addition of sodium bicarbonate. The product is subsequently extracted with dichloromethane. The organic phase is dried over sodium sulphate and the solvent is evaporated under reduced pressure. The solid residue is purified on 35 g of silica gel, eluting with a mixture of dichloromethane, methanol and aqueous ammonia (98/2/0.2), to give 0.77 g of a white solid.

m.p.: 132-134° C.

¹H NMR (CDCl₃) δ: 8.35 (d, 1H), 7.90 (d, 1H), 7.3 (d, 1H), 7.25 (s, 1H), 7.15 (d, 1H), 4.05 (s, 3H), 2.65 (s, 3H) ppm.

Step 4.2. (−)-3-(2-Methoxypyridin-4-yl)-2-methyl-6-((R)-3-methylpiperazin-1-yl)imidazo[1,2-b]-pyridazine

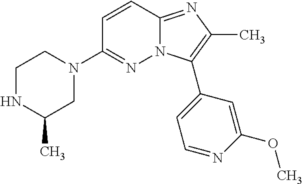

A solution of 0.35 g (1.3 mmol) of 6-chloro-2-methyl-3-(2-methoxypyridin-4-yl)imidazo[1,2-b]-pyridazine, obtained in step 4.1., and 0.38 g (3.8 mmol) of (R)-2-methylpiperazine in 5 ml of pentanol is heated at 150° C. for 24 hours. Then a further 0.1 g (1.0 mmol) of (R)-2-methylpiperazine is added and heating is continued for 24 hours. The mixture is poured into 1N aqueous hydrochloric acid solution. The aqueous phase is washed with ethyl acetate and then basified using sodium bicarbonate solution, and the product is extracted with dichloromethane. The organic phase is dried over sodium sulphate and the solvent is stripped off under reduced pressure. The oily residue is purified on 25 g of silica gel, eluting with a mixture of dichloromethane, methanol and aqueous ammonia (95/5/0.5), to give 0.15 g of a white solid.

m.p.: 104-106° C.

α^{D} (c=1; CH₂Cl₂)=−17.1°

¹H NMR (CDCl₃) δ: 8.25 (d, 1H), 7.70 (d, 1H), 7.40 (d, 1H), 7.25 (s, 1H), 6.85 (d, 1H), 3.9-4.1 (m, 2H), 4.00 (s, 3H), 2.85-3.2 (m, 4H), 2.60 (s, 3H), 2.6 (m, 1H), 2.0 (broad m, 1H), 1.15 (d, 3H) ppm.

### EXAMPLE 5

### Compound 68

### {4-[6-(Hexahydropyrrolo[3,4-c]pyrrol-2(1H)-yl)-2-methylimidazo[1,2-b]pyridazin-3-yl]pyridin-2-yl}amine

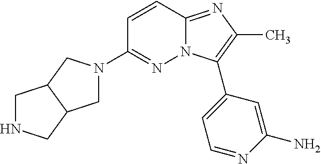

Step 5.1.: tert-Butyl [4-(4,4,5,5-tetramethyl-1,3,2-dioxaborolan-2-yl)pyridin-2-yl]carbamate

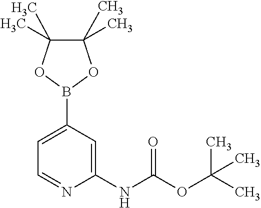

A solution of 6.76 g (24.8 mmol) of tert-butyl (4-bromopyridin-2-yl)carbamate (Deady, Leslie W.; Korytsky, Olga L.; Rowe, Jeffrey E.; Aust. J. Chem.; 35; 10; 1982; 2025-2034) in 150 ml of dimethylformamide is admixed with 8.0 g (81 mmol) of potassium acetate, dried at 130° C. beforehand, and with 6.9 g (27 mmol) of bis(pinacolato)diboron. Subsequently a stream of argon is bubbled in for a few moments, and 1.2 g (1.5 mmol) of 1,1′-bis(diphenylphosphino)ferrocenedichloropalladium(II) are added. The mixture is stirred at 80° C. under argon for 2 hours and then poured into saturated aqueous ammonium chloride solution. The product is extracted with ethyl acetate, the organic phase is dried over sodium sulphate and the solvent is stripped off under reduced pressure. The residue is triturated in 300 ml of diisopropyl ether at reflux and the insoluble matter is separated by filtration.

The filtrate is cooled and partially concentrated under reduced pressure. Following addition of 70 ml of hexane, the precipitate formed is isolated by filtration, to give 4.2 g of an orange solid after drying.

m.p.: 188-193° C.

¹H NMR (CDCl₃) δ: 8.15 (m, 2H), 7.85 (broad s, 1H), 7.15 (d, 1H), 1.40 (s, 9H), 1.20 (s, 12H) ppm.

Step 5.2. tert-Butyl [4-(6-chloro-2-methylimidazo[1,2-b]pyridazin-3-yl)pyridin-2-yl]carbamate

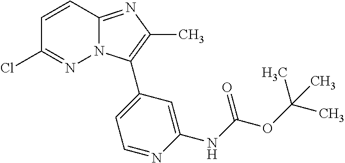

A mixture of 3.19 g (10.9 mmol) of 6-chloro-3-iodo-2-methylimidazo[1,2-b]pyridazine, 4.18 g (13.0 mmol) of tert-butyl [4-(4,4,5,5-tetramethyl-1,3,2-dioxaborolan-2-yl)pyridin-2-yl]-carbamate and 10.6 g (32.6 mmol) of caesium carbonate in 250 ml of a mixture of tetrahydrofuran and water (90/10) is purged with argon. Then 0.80 g (0.98 mmol) of 1,1′-bis(diphenylphosphino)ferrocenedichloropalladium(II) (PdCl₂ (dppf)) is added. The reaction is heated for 3 hours at reflux and then the solvent is evaporated under reduced pressure. The residue is taken up with chloroform and the organic phase is washed with saturated aqueous ammonium chloride solution. The organic phase is dried over sodium sulphate and the solvent is evaporated under reduced pressure. The solid residue is purified on 110 g of silica gel, eluting with a mixture of dichloromethane, methanol and aqueous ammonia (97/3/0.3), to give 3.6 g of a dark beige solid. The solid is triturated in diethyl ether to give 3.0 g of white solid after filtration and drying.

m.p.: 260° C.

¹H NMR (CDCl₃) δ: 8.50 (d, 1H), 8.45 (s, 1H), 7.90 (d, 1H), 7.85 (broad s, 1H), 7.45 (d, 1H), 7.10 (d, 1H), 2.70 (s, 3H), 1.55 (s, 9H) ppm.

Step 5.3. 4-(6-Chloro-2-methylimidazo[1,2-b]pyridazin-3-yl)pyridin-2-ylamine

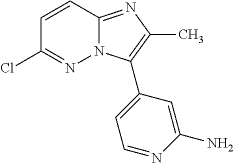

A solution of 7.70 g (21.5 mmol) of tert-butyl [4-(6-chloro-2-methylimidazo[1,2-b]pyridazin-3-yl)pyridin-2-yl]carbamate, obtained in step 5.2., in 200 ml of chloroform is admixed with 80 ml (1.1 mol) of trifluoroacetic acid at 100° C. The mixture is stirred at ambient temperature for 4 hours and then the solvent is evaporated under reduced pressure. The resulting brown oil is taken up with 3N aqueous hydrochloric acid solution and the aqueous phase is washed with diethyl ether.

The aqueous phase is subsequently alkalified by addition of dilute aqueous ammonia, and the precipitate which forms is separated by filtration. The solid is dissolved in chloroform and the solution is washed with water, dried over sodium sulphate and concentrated under reduced pressure, to give a white solid. This solid is triturated in a mixture of diethyl ether and hexane, to give 3.9 g of white powder after filtration and drying.

Yield: 70%

m.p.: 188° C.

¹H NMR (CDCl₃) δ: 8.25 (d, 1H), 7.9 (d, 1H), 7.10 (d, 1H), 7.05 (d, 1H), 6.95 (s, 1H), 4.7 (sl, 2H), 2.65 (s, 3H) ppm.

Step 5.4. {4-[6-(5-Benzylhexahydropyrrolo[3,4-c]pyrrol-2-yl)-2-methylimidazo[1,2-b]pyridazin-3-yl]-pyridin-2-yl}amine

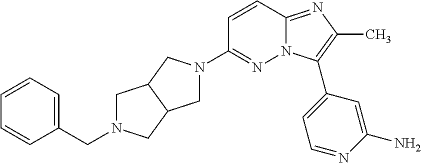

In a sealed tube, a solution of 0.62 g (2.4 mmol) of 4-(6-chloro-2-methylimidazo[1,2-b]-pyridazin-3-yl)pyridin-2-ylamine, obtained in step 5.3., and 1.5 g (7.3 mol) of 2-benzyloctahydropyrrolo[3,4-c]pyrrole in 5 ml of pentanol is heated at 150° C. for 40 hours. After cooling, the mixture is treated with 1N aqueous hydrochloric acid solution. The resulting aqueous phase is washed with diethyl ether and then basified using dilute aqueous ammonia.

The product is extracted with dichloromethane and the organic phase is dried over sodium sulphate and concentrated under reduced pressure.

The brown oil isolated is chromatographed on a silica gel column, eluting with a mixture of dichloromethane, methanol and aqueous ammonia (95/5/0.5), to give 0.89 g of amorphous solid.

Yield: 87%

¹H NMR (CDCl₃) δ: 8.20 (d, 1H), 7.65 (d, 1H), 7.3 (m, 5H), 7.20 (d, 1H), 7.05 (s, 1H), 6.65 (d, 1H), 4.5 (bs, 2H), 3.8-3.55 (m, 2H and s, 2H), 3.35 (dd, 2H), 3.00 (m, 2H), 2.75 (m, 2H), 2.60 (s, 3H), 2.55 (dd, 2H) ppm.

Step 5.5. {4-[6-(Hexahydropyrrolo[3,4-c]pyrrol-2(1H)-yl)-2-methylimidazo[1,2-b]pyridazin-3-yl]pyridin-2-yl}amine

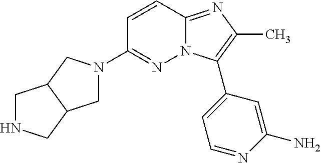

A mixture of 0.88 g (2.1 mmol) of {4-[6-(5-benzylhexahydropyrrolo[3,4-c]pyrrol-2(1H)-yl)-2-methylimidazo[1,2-b]pyridazin-3-yl]pyridin-2-yl}amine obtained in step 5.4., 2.0 g (31 mmol) of ammonium formate and 1.0 g of 10% palladium on carbon (50% moisture content) in 60 ml of methanol is stirred at reflux for one hour. The mixture is cooled and filtered. The filtrate is concentrated under reduced pressure and the residue is taken up in dichloromethane. The organic phase is washed with a minimum of 1 N sodium hydroxide solution, dried over sodium sulphate and concentrated under reduced pressure. The residue is purified by chromatography on silica gel, eluting with a mixture of dichloromethane, methanol and aqueous ammonia (90/10/1), to give 0.17 g of beige solid after trituration in a mixture of 20 ml of diethyl ether and 5 ml of acetonitrile, filtration and drying.

m.p. >155° C. (decomposition); M+H: 336

¹H NMR (CDCl₃) δ: 8.20 (d, 1H), 7.65 (d, 1H), 7.20 (d, 1H), 7.05 (s, 1H), 6.65 (d, 1H), 4.5 (bs, 1H), 3.7 (m, 2H), 3.5-3.1 (m, 4H), 3.1-2.8 (m, 4H), 2.60 (s, 3H), 2 (bs, 2H) ppm.

### EXAMPLE 6

### Compound 69

### {4-[6-(Hexahydropyrrolo[3,4-c]pyrrol-2(1H)-yl)-2-methyl-imidazo[1,2-b]pyridazin-3-yl]pyridin-2-yl}methylamine

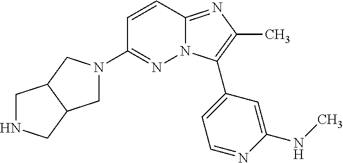

Step 6.1. tert-Butyl [4-(6-chloro-2-methylimidazo[1,2-b]pyridazin-3-yl)pyridin-2-yl]methylcarbamate

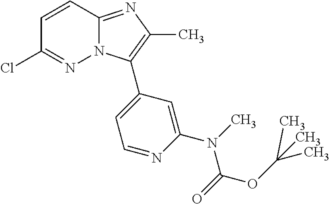

A suspension of 0.22 g (5.6 mmol) of sodium hydride (at 60% in oil) in 35 ml of dimethylformamide, cooled at 00° C. and under a stream of argon, is admixed in portions with 1.6 g (4.5 mmol) of tert-butyl [4-(6-chloro-2-methylimidazo[1,2-b]pyridazin-3-yl)pyridin-2-yl]-carbamate. The mixture is stirred for 40 minutes at between 0 and 10° C. and then admixed dropwise with 0.73 g (5.2 mmol) of methyl iodide in dilution in dimethylformamide. After 18 hours at ambient temperature, the mixture is poured into saturated aqueous ammonium chloride solution and the product is extracted with ethyl acetate. The organic phase is dried over sodium sulphate and concentrated under reduced pressure, to give a brown oil which is chromatographed on a silica gel column, eluting with a mixture of dichloromethane, methanol and aqueous ammonia (98/2/0.2). Recrystallization from hexane, filtration and drying thus give 1.1 g of beige powder,

m.p.: 117-120° C.

¹H NMR (CDCl₃) δ: 8.55 (d, 1H), 8.1 (s, 1H), 7.90 (d, 1H), 7.45 (d, 1H), 7.1 (d, 1H), 3.50 (s, 3H), 2.70 (s, 3H), 1.55 (s, 9H) ppm.

Step 6.2. [4-(6-Chloro-2-methylimidazo[1,2-b]pyridazin-3-yl)pyridin-2-yl]methylamine

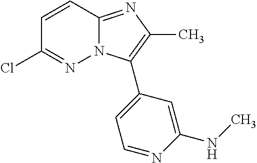

A solution of 1.03 g (2.76 mmol) of tert-butyl [4-(6-chloro-2-methylimidazo[1,2-b]pyridazin-3-yl)pyridin-2-yl]methylcarbamate, obtained in step 6.1., in 20 ml of chloroform is admixed with ml (270 mmol) of trifluoroacetic acid at 10° C. The mixture is stirred at ambient temperature for 18 hours and then the solvent is evaporated under reduced pressure. The resulting brown oil is triturated with 30 ml of diethyl ether. The solid form is separated by filtration. It is subsequently dissolved in chloroform and the solution is washed with dilute aqueous ammonia, dried over sodium sulphate and concentrated under reduced pressure, to give 0.70 g of beige powder after drying.

m.p.: 268-274° C.

¹H NMR (CDCl₃) δ: 8.25 (d, 1H), 7.85 (d, 1H), 7.10 (d, 1H), 6.95 (d, 1H), 6.85 (d, 1H), 4.85 (bs, 1H), 3.00 (d, 3H), 2.65 (s, 3H) ppm.

Step 6.3. {4-[6-(5-Benzylhexahydropyrrolo[3,4-c]pyrrol-2-yl)-2-methylimidazo[1,2-b]pyridazin-3-yl]pyridin-2-yl}methylamine

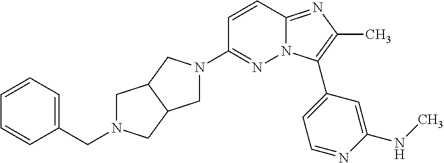

In a sealed tube, a solution of 0.67 g (2.5 mmol) of [4-(6-chloro-2-methylimidazo[1,2-b]-pyridazin-3-yl)pyridin-2-yl]methylamine, obtained in step 6.2., and 1.5 g (7.3 mmol) of 2-benzyloctahydropyrrolo[3,4-c]pyrrole in 5 ml of pentanol is heated at 150° C. for 40 hours. After cooling, the mixture is treated with 1N aqueous hydrochloric acid solution. The resulting aqueous phase is washed with diethyl ether and then basified using dilute aqueous ammonia.

The product is extracted with dichloromethane and the organic phase is dried over sodium sulphate and concentrated under reduced pressure.

The brown oil isolated is chromatographed on a silica gel column, eluting with a mixture of dichloromethane, methanol and aqueous ammonia (95/5/0.5), to give 0.98 g of amorphous solid.

¹H NMR (CDCl₃) δ: 8.20 (d, 1H), 7.65 (d, 1H), 7.3 (m, 5H), 7.10 (d, 1H), 7.05 (s, 1H), 6.65 (d, 1H), 4.7 (bd, 1H), 3.8-3.55 (m, 2H and s, 2H), 3.4 (dd, 2H), 2.95 (m, 5H), 2.75 (m, 2H), 2.65 (s, 3H), 2.55 (dd, 2H) ppm.

Step 6.4. {4-[6-(Hexahydropyrrolo[3,4-c]pyrrol-2(1H)-yl)-2-methylimidazo[1,2-b]pyridazin-3-yl]pyridin-2-yl}methylamine

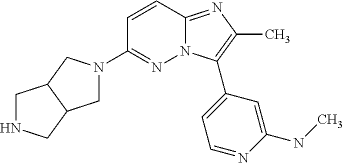

A mixture of 0.97 g (2.2 mmol) of {4-[6-(5-benzylhexahydropyrrolo[3,4-c]pyrrol-2(1H)-yl)-2-methylimidazo[1,2-b]pyridazin-3-yl]pyridin-2-yl}methylamine, obtained in step 6.4., 2.1 g (33 mmol) of ammonium formate and 1.0 g of 10% palladium on carbon (50% moisture content) in 60 ml of methanol is stirred at reflux for one hour. The mixture is cooled and filtered. The filtrate is concentrated under reduced pressure and the residue is taken up in dichloromethane. The organic phase is washed with a minimum of 1N sodium hydroxide solution, dried over sodium sulphate and concentrated under reduced pressure. The residue is purified by chromatography on silica gel, eluting with a mixture of dichloromethane, methanol and aqueous ammonia (90/10/1), to give 0.40 g of beige solid (containing 20 mol % of ether) after trituration in a mixture of 20 ml of diethyl ether and 5 ml of acetonitrile, filtration and drying.

m.p.: 169° C. (degradation)

¹H NMR (CDCl₃) δ: 8.20 (d, 1H), 7.65 (d, 1H), 7.10 (d, 1H), 7.0 (s, 1H), 6.65 (d, 1H), 4.65 (bs, 1H), 3.7 (m, 2H), 3.4 (m, 2H), 3.2 (m, 2H), 3.05-2.8 (d, 3H and m, 4H), 2.60 (s, 3H) ppm.

### EXAMPLE 7

### Compound 79

### 9-(2-Methyl-3-pyridin-4-ylimidazo[1,2-b]pyridazin-6-yl)-1=oxa-4,9-diazaspiro[5.5]undecane hydrochloride (3:1)

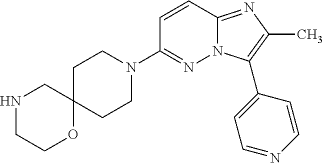

Step 7.1. 6-Chloro-3-(pyridin-4-yl)imidazo[1,2-b]pyridazine

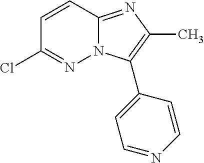

A solution of 8.90 g (53.1 mmol) of 6-chloro-2-methylimidazo[1,2-b]pyridazine in 270 ml of dimethylformamide is admixed with 13.1 g (63.7 mmol) of 4-iodopyridine and 7.34 g (53.1 mmol) of potassium carbonate. Argon is then bubbled in and 0.60 g (2.7 mmol) of palladium(II) acetate is added. The mixture is heated at 135° C. for 3 hours and then poured into water, and the product is extracted with ethyl acetate. The organic phase is dried over sodium sulphate and the solvent is evaporated under reduced pressure. The resulting yellow solid is organized with stirring for 30 minutes in acetonitrile, and 6 g of a dark beige solid are isolated. The mother liquors are concentrated under reduced pressure and purified on 110 g of silica gel, eluting with a mixture of dichloromethane, methanol and aqueous ammonia (98/2/0.2), to give 1.5 g of additional product, whose purity is equivalent to that of the first fraction.

Overall yield: 58%

m.p.: 180° C.

¹H NMR (CDCl₃) δ: 8.60 (d, 2H), 7.80 (d, 1H), 8.55 (d, 2H), 7.95 (d, 1H), 2.50 (s, 3H)

Step 7.2. tert-Butyl 9-(2-methyl-3-pyridin-4-ylimidazo[1,2-b]pyridazin-6-yl)-1-oxa-4,9-diazaspiro[5.5]undecane-4-carboxylate

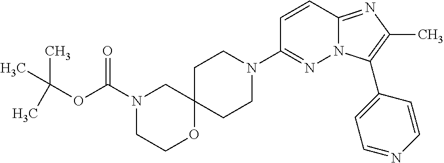

In a reactor a solution of 0.50 g (2.0 mmol) of 6-chloro-3-(pyridin-4-yl)imidazo[1,2-b]-pyridazine, obtained in step 7.1., 0.90 g (3.1 mmol) of tert-butyl 1-oxa-4,9-diazaspiro[5.5]-undecane-4-carboxylate and 0.84 ml (5.1 mmol) of diisopropylethylamine in 5 ml of pentanol is heated at 150° C. for 24 hours. After cooling, the pentanol is removed by evaporation under reduced pressure and the residue is taken up with aqueous sodium bicarbonate solution. The product is extracted with dichloromethane. The organic phase is dried over sodium sulphate and concentrated under reduced pressure and the residue is chromatographed, eluting with a mixture of dichloromethane, methanol and aqueous ammonia (97/3/0.3), to give 0.17 g of orange oil, which is used as it is in the remainder of the synthesis.

Step 7.3. 9-(2-methyl-3-pyridin-4-ylimidazo[1,2-b]pyridazin-6-yl)-1-oxa-4,9-diazaspiro[5.5]-undecane hydrochloride (3:1)

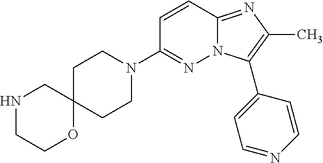

A solution of 0.17 g (0.37 mmol) of tert-butyl 9-(2-methyl-3-pyridin-4-ylimidazo[1,2-b]-pyridazin-6-yl)-1-oxa-4,9-diazaspiro[5.5]undecane-4-carboxylate, obtained in step 7.2., in ml of dichloromethane is admixed with 0.55 ml of trifluoroacetic acid and the solution is stirred at ambient temperature for one hour.

The acid is then neutralized by addition of aqueous sodium bicarbonate solution, and the organic phase is separated and dried over sodium sulphate. Following concentration under reduced pressure, the residue is chromatographed, eluting with a mixture of dichloromethane, methanol and aqueous ammonia (90/10/1), to give a yellow oil.

The oil is taken up in acetone and the product is converted to its trihydrochloride by addition of a 5-6N aqueous solution of hydrochloric acid in isopropanol. The solvents are evaporated under reduced pressure and the solid residue is triturated in ethanol, to give 180 mg of beige powder.

m.p. >255° C.; M+H: 365

¹H NMR (DMSO d₆) δ: 9.7 (bs, 2H), 8.90 (d, 2H), 8.35 (d, 2H), 8.10 (d, 1H), 7.65 (d, 1H), 5.3 (bs), 3.9 (m, 4H), 3.3 (m, 2H), 3.0 (m, 4H), 2.65 (m, 3H), 2.1 (m, 2H), 1.7 (m, 2H)

### EXAMPLE 8

### Compound 70

### 2-Methyl-6-(5-methylhexahydropyrrolo[3,4-c]pyrrol-2(1H)-yl)-3-pyridin-4-ylimidazo[1,2-b]pyridazine

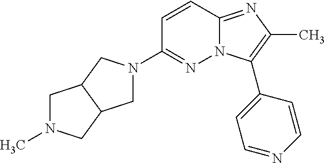

In a reactor, a solution of 0.40 g (1.6 mmol) of 6-chloro-3-(pyridin-4-yl)imidazo[1,2-b]-pyridazine, obtained in step 7.1., 0.41 g (3.3 mmol) of 2-methylhexahydropyrrolo[3,4-c]pyrrole and 0.27 ml (1.6 mmol) of diisopropylethylamine in 5 ml of pentanol is heated at 150° C. for 4 days. After cooling, the mixture is treated with 1N aqueous hydrochloric acid solution. The resulting aqueous phase is washed with diethyl ether and then basified using dilute aqueous ammonia.

The product is extracted with dichloromethane and the organic phase is dried over sodium sulphate and concentrated under reduced pressure.

The brown oil isolated is chromatographed on a silica gel column, eluting with a mixture of dichloromethane, methanol and aqueous ammonia (95/5/0.5), to give 0.148 g of white powder following crystallization from diethyl ether and drying.

m.p.: 155-159° C.

¹H NMR (DMSO d₆) δ: 8.70 (d, 2H), 7.85 (d, 2H), 7.70 (d, 1H), 6.70 (d, 1H), 3.80 (m, 2H), 3.40 (m, 2H), 3.05 (m, 2H), 2.70 (m, 2H), 2.65 (s, 3H), 2.60 (m, 2H), 2.40 (s, 3H)

### EXAMPLE 9

### Compound No. 71

### 2,7-Dimethyl-6-(5-ethylhexahydropyrrolo[3,4-c]pyrrol-2(1H)-yl)-3-pyridin-4-ylimidazo[1,2-b]pyridazine

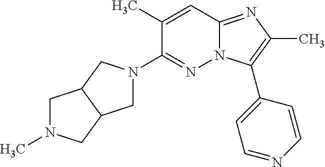

Step 9.1. 6-Chloro-4-methylpyridazin-3-ylamine and 6-chloro-5-methylpyridazin-3-ylamine

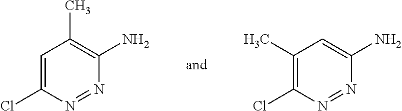

A mixture of 50.0 g (307 mmol) of 3,6-dichloro-4-methylpyridazine in 170 ml of aqueous ammonia (30%) is heated at 120° C. for 16 h in a steel reactor at the internal pressure of bar.

The reactor is cooled and the reaction mixture is poured into 200 ml of water. The solid formed is isolated by filtration and dried under vacuum, to give 38.5 g of a mixture containing approximately 45% of 6-chloro-4-methylpyridazin-3-ylamine (CAS 64068-00-4) and 55% of 6-chloro-5-methylpyridazin-3-ylamine (CAS 66346-87-0).

¹H NMR (CDCl₃) δ: 7.20 and 6.75 (2s, 1H): (d, 0.55H), 4.9 (sl, 2H), 2.40 and 2.25 (2s, 3H) ppm.

Step 9.2. 6-Chloro-2,8-dimethylimidazo[1,2-b]pyridazine and 6-chloro-2,7-dimethylimidazo[1,2-b]pyridazine

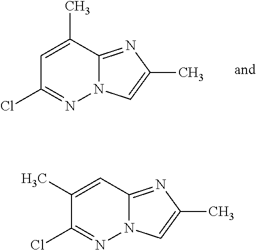

The mixture of 16.2 g (174 mmol) of 2-bromoacetone (CAS 78-95-5) with 19.3 g (134 mmol) of the mixture of 6-chloro-4-methylpyridazin-3-ylamine and 6-chloro-5-methylpyridazin-3-ylamine obtained in Step 9.1 in 200 ml of n-butanol is heated at 120° C. for 18 hours. After cooling, the solvent is removed by evaporation under reduced pressure and the solid is triturated in 170 ml of acetone. After freezing, the solid is separated by filtration. The dark beige powder is taken up in chloroform and rendered basic by addition of ammonia solution. The product is extracted with chloroform, the organic phase is dried over sodium sulphate and the solvent is evaporated under reduced pressure to give 14 g of a brown solid. The two isomers are separated by chromatography on an alumina column (800 g). The product is deposited on the column in solution in a mixture of toluene and dichloromethane, and the isomers are then separated by eluting by a gradient of cyclohexane in dichloromethane (50% to 0%). This produces, in succession, 5.2 g of 6-chloro-2,8-dimethylimidazo[1,2-b]pyridazine and 6.0 g of 6-chloro-2,7-dimethylimidazo[1,2-b]pyridazine in the form of white powders after trituration in 50 ml of diisopropyl ether, filtration and drying.

6-Chloro-2,8-dimethylimidazo[1,2-b]pyridazine:

m.p.: 117-119° C.

¹H NMR (CDCl₃) δ: 8.05 (s, 1H), 2.55 (s, 3H), 2.40 (s, 3H) ppm.

6-Chloro-2,7-dimethylimidazo[1,2-b]pyridazine:

m.p.: 185-188° C.

¹H NMR (CDCl₃) δ: 8.00 (s and s, 2H), 2.40 (s and s, 6H) ppm.

Step 9.3. 6-Chloro-2,7-dimethyl-3-iodoimidazo[1,2-b]pyridazine

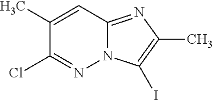

A solution of 6.00 g (33.0 mmol) of 6-chloro-2,7-dimethylimidazo[1,2-b]pyridazine in 100 ml of chloroform at ambient temperature is admixed rapidly with 82.6 ml (82.6 mmol) of a 1 M solution of iodine monochloride in dichloromethane. The reaction is then left at ambient temperature for an hour and then aqueous sodium bicarbonate solution and 5% aqueous sodium thiosulphate solution are added until decolouring is achieved. The product is extracted with dichloromethane, the organic phase is dried over sodium sulphate and the solvent is evaporated under reduced pressure.

The yellowish solid residue is triturated in 50 ml of diisopropyl ether and then isolated by filtration to give 9.7 g of a yellow powder after drying.

m.p.: 219-220° C.

¹H NMR (CDCl₃) δ: 7.70 (s, 1H), 2.60 (s, 3H), 2.55 (s, 3H) ppm.

Step 9.4. 6-Chloro-2,7-dimethyl-3-(pyridin-4-yl)imidazo[1,2-b]pyridazine

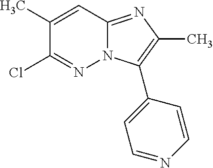

A solution of 4.82 g (15.7 mmol) of 6-chloro-2,7-dimethyl-3-iodoimidazo[1,2-b]pyridazine, 2.72 g (18.8 mmol) of pyridin-4-ylboronic acid and 15.3 g (47 mmol) of caesium carbonate in 220 ml of a mixture of tetrahydrofuran and water (9:1), under argon, is admixed with 1.15 g (1.41 mmol) of a complex of 1,1′-bis(diphenylphosphino)ferrocenedichloropalladium(II) and dichloromethane (PdCl₂(dppf).CH₂Cl₂). After 18 hours of heating at reflux, the mixture is poured into ice-cold aqueous 1 N hydrochloric acid, and the aqueous phase is washed with ethyl acetate and then rendered basic by addition of sodium bicarbonate. The product is subsequently extracted with dichloromethane. The organic phase is dried over sodium sulphate and the solvent is evaporated under reduced pressure. The solid residue is purified on 120 g of silica gel, eluting with a mixture of dichloromethane, methanol and aqueous ammonia (96/4/0.4), to give 3.05 g of a white solid after trituration in diisopropyl ether, filtration and drying.

m.p.: 178-181° C.

¹H NMR (DMSO de) δ: 8.75 (d, 2H), 8.17 (s, 1H), 7.80 (d, 2H), 2.60 (s, 3H), 2.45 (s, 3H) ppm

Step 9.5. 2,7-Dimethyl-6-(5-methylhexahydropyrrolo[3,4-c]pyrrol-2(H)-yl)-3-pyridin-4-ylimidazo[1,2-b]pyridazine

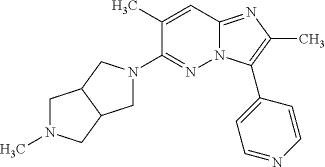

In a reactor a solution of 0.357 g (1.45 mmol) of 6-chloro-2,7-dimethyl-3-(pyridin-4-yl)imidazo[1,2-b]pyridazine, 0.256 g (2.03 mmol) of 2-methylhexahydropyrrolo[3,4-c]pyrrole and 0.20 ml (1.5 mmol) of triethylamine in 4 ml of pentanol is heated at 150° C. for 3 days. After cooling, the mixture is treated with aqueous 1N hydrochloric acid solution. The aqueous phase obtained is washed with diethyl ether and then rendered basic using dilute aqueous ammonia.

The product is extracted with dichloromethane and the organic phase is dried over sodium sulphate and concentrated under reduced pressure.

The brown oil isolated is chromatographed on a silica gel column, eluting with a mixture of dichloromethane, methanol and aqueous ammonia (93/7/0.7), to give 0.230 g of white powder after recrystallization in acetonuria and drying.

m.p.: 139-142° C.

¹H NMR (DMSO d₆) δ: 8.70 (d, 2H), 7.85 (d, 2H), 7.75 (s, 1H), 3.80 (m, 2H), 3.45 (dd, 2H), 3.25 (dd, 2H), 2.85 (m, 2H), 2.65 (dd, 2H), 2.55 (s, 3H), 2.45 (s, 3H), 2.35 (dd, 3H), 2.25 (s, 3H).

Table 1 below illustrates the chemical structures and the physical properties of some compounds according to the invention.

In this table:
the column “m.p. ° C.” reports the melting points of the products in degrees Celsius. “N.D” means that the melting point is not determined;
in the “salt” column, “HCl” represents a compound in hydrochloride form, and the ratio between brackets is the acid:base ratio, “HBr” represents a compound in hydrobromide form and the ratio between brackets is the acid:base ratio, and the sign “—” signifies that the compound is in base form;
the [α]_{D} column reports the result of analysis of the optical rotation of the compounds from the table at a wavelength of 589 nm; the solvent indicated in brackets corresponds to the solvent used to carry out the measurement of the optical rotation in degrees, and the letter “C” indicates the concentration of the solvent in g/100 ml. “N.A.” signifies that the optical rotation measurement is not applicable,
the column “LC-MS or (MS)” reports the result of analysis of the products by LC-MS (liquid chromatography coupled to Mass Spectroscopy), carried out on an Agilent LC-MSD Trap instrument in positive ESI mode, or by MS (Mass Spectroscopy) on an Autospec M (EBE) instrument, utilizing the DC-INH₃ technique;
decomp. signifies decomposition;
degrad. signifies degradation,
“F” means fluoro,
“Cl” means chloro,
“CH₃—” means methyl,
“C₂H₅—” means ethyl,
“CH₃O—” means methoxy,
“CH₃OH” means methanol;
“CH₃—O—CH₂—” means methoxymethyl,
“c-Propyl-” means cyclopropyl,
“c-Butyl-” means cyclobutyl,
“c-Propyl-CH₂—” means cyclopropylmethyl,
“NH₂—” means amino,
“CH₃NH—” means methylamino,
“CH₂Cl₂” means dichloromethane,

TABLE 1

| | | | | | | | [α]_{D} (°) | | |
|---|---|---|---|---|---|---|---|---|---|
| | | | | | | | (solvent; C | | |
| | | | | | | | in | | |
| No | NALB | R₇ | R₈ | R₂ | R₃ | Salt | g/100 ml) | m.p. °C. | M + H |
| 1 | Piperazin-1-yl | H | H | CH₃— | H | — | N.A. | 166-168 | 295 |
| 2 | Piperazin-1-yl | H | H | CH₃— | H | HCl (3:1) | N.A. | 319-321 | 295 |
| 3 | Piperazin-1-yl | H | H | CH₃— | F | — | N.A. | 139-141 | 313 |
| 4 | Piperazin-1-yl | CH₃— | CH₃— | CH₃— | H | HCl (3:1) | N.A. | N.D. | 323 |
| 5 | Piperazin-1-yl | H | H | CH₃—O—CH₂— | H | | N.A. | N.D. | 325 |
| 6 | Piperazin-1-yl | H | H | C₂H₅— | H | — | N.A. | N.D. | 309 |
| 7 | Piperazin-1-yl | H | H | C₂H₅— | H | HCl (3:1) | N.A. | N.D. | 309 |
| 8 | Piperazin-1-yl | H | H | C₂H₅— | CH₃— | — | N.A. | N.D. | 323 |
| 9 | Piperazin-1-yl | H | H | Isopropyl- | H | — | N.A. | 199-201 | 323 |
| 10 | Piperazin-1-yl | H | H | Isopropyl- | CH₃— | — | N.A. | N.D. | 337 |
| 11 | Piperazin-1-yl | H | H | c-Propyl- | H | — | N.A. | 169-171 | 321 |
| 12 | Piperazin-1-yl | H | H | c-Propyl- | CH₃— | — | N.A. | N.D. | 335 |
| 13 | Piperazin-1-yl | H | H | c-Propyl- | NH₂— | — | N.A. | N.D. | 336 |
| 14 | Piperazin-1-yl | H | H | c-Propyl- | CH₃O— | — | N.A. | N.D. | 351 |
| 15 | Piperazin-1-yl | H | H | c-Propyl- | Cl | — | N.A. | N.D. | 355 |
| 16 | Piperazin-1-yl | H | H | Isobutyl- | H | — | N.A. | 92-119 | 337 |
| 17 | Piperazin-1-yl | H | H | Isobutyl- | H | HCl (3:1) | N.A. | N.D. | 337 |
| 18 | Piperazin-1-yl | H | H | c-Propyl-CH₂— | H | — | N.A. | N.D. | 335 |
| 19 | Piperazin-1-yl | H | H | c-Butyl- | H | — | N.A. | 189-191 | 335 |
| 20 | (R,S)-3-Methylpiperazin-1-yl | H | H | CH₃— | H | — | N.A. | 133-135 | 309 |
| 21 | (R,S)-3-Methylpiperazin-1-yl | H | H | CH₃— | CH₃— | — | N.A. | 145-147 | 323 |
| 22 | (R,S)-3-Methylpiperazin-1-yl | H | H | c-Propyl- | CH₃— | — | N.A. | N.D. | 349 |
| 23 | (R,S)-3-Methylpiperazin-1-yl | H | H | c-Propyl- | NH₂— | — | N.A. | N.D. | 350 |
| 24 | (R,S)-3-Methylpiperazin-1-yl | H | H | c-Propyl- | CH₃O— | — | N.A. | N.D. | 365 |
| 25 | (R,S)-3-Methylpiperazin-1-yl | H | H | c-Propyl- | Cl | — | N.A. | N.D. | 369 |
| 26 | (R)-3-Methylpiperazin-1-yl | H | H | CH₃— | H | — | −18.3 (CH₃OH; | 146-148 | 309 |
| | | | | | | | C = 1.012) | 150-152 | |
| 27 | (R)-3-Methylpiperazin-1-yl | H | H | CH₃— | CH₃— | — | +3.3 (CH₂Cl₂; | 157-159 | 323 |
| | | | | | | | C = 1.032) | | |
| 28 | (R)-3-Methylpiperazin-1-yl | H | H | CH₃— | NH₂— | — | −11.6 (CH₃OH; | 172-177 | 324 |
| | | | | | | | C = 1.000) | 174-177 | |
| 29 | (R)-3-Methylpiperazin-1-yl | H | H | CH₃— | CH₃NH— | — | −6.6 (CH₃OH; | 65 | 338 |
| | | | | | | | C = 0.966) | | |
| 30 | (R)-3-Methylpiperazin-1-yl | H | H | CH₃— | CH₃O— | — | −17.1 (CH₂Cl₂; | 104-106 | 339 |
| | | | | | | | C = 1.000) | | |
| 31 | (R)-3-Methylpiperazin-1-yl | H | H | C₂H₅— | H | — | −4.3 (CHCl₃; | N.D. | 323 |
| | | | | | | | C = 0.235). | | |
| 32 | (R)-3-Methylpiperazin-1-yl | H | H | C₂H₅— | F | — | −13.9 (CHCl₃; | N.D. | 341 |
| | | | | | | | C = 0.245). | | |
| 33 | (R)-3-Methylpiperazin-1-yl | H | H | c-Propyl-CH₂— | H | — | −20.2 (CHCl₃; | N.D. | 349 |
| | | | | | | | (C = 0.104) | | |
| 34 | (R)-3-Methylpiperazin-1-yl | H | H | c-Propyl-CH₂— | CH₃— | — | −9.6 (CHCl₃; | N.D. | 363 |
| | | | | | | | C = 0.114) | | |
| 35 | (S)-3-Methylpiperazin-1-yl | H | H | CH₃— | H | — | +17.3 (CH₃OH; | 146-148 | 309 |
| | | | | | | | C = 0.788) | | |
| 36 | (S)-3-Methylpiperazin-1-yl | H | H | CH₃— | CH₃O— | — | +16.3 (CH₂Cl₂; | 106-108 | 339 |
| | | | | | | | C = 1.000) | | |
| 37 | (S)-3-Methylpiperazin-1-yl | H | H | c-Propyl- | H | — | −12.3 (CHCl₃; | N.D. | 335 |
| | | | | | | | C = 0.77) | | |
| 38 | (S)-3-Methylpiperazin-1-yl | H | H | CH₃— | NH₂— | — | +11.7 (CH₂Cl₂; | 172-177 | 324 |
| | | | | | | | C = 1.000) | 177-178 | |
| 39 | 4-Methylpiperazin-1-yl | H | H | CH₃— | H | — | N.A. | 150-152 | 309 |
| | | | | | | | | 154-156 | |
| 40 | 4-Methylpiperazin-1-yl | H | H | CF₃— | H | — | N.A. | 182-184 | 363 |
| 41 | (R,S)-3-Hydroxymethylpiperazin-1-yl | H | H | CH₃— | H | — | N.A. | 237-240 | 325 |
| 42 | 4-Ethylpiperazin-1-yl | H | H | CH₃— | H | HCl (3:1) | N.A. | 305-307 | 323 |
| 43 | 4-Ethylpiperazin-1-yl | H | H | CH₃— | CH₃— | — | N.A. | 144-146 | 337 |
| 44 | 4-(iso-Propyl)piperazin-1-yl | H | H | CH₃— | H | HCl (3:1) | N.A. | 304-306 | 338 |
| 45 | 4-(iso-Propyl)piperazin-1-yl | H | H | CH₃— | CH₃— | — | N.A. | 97-99 | 351 |
| 46 | 4-(iso-Propyl)piperazin-1-yl | H | H | CH₃— | NH₂— | HCl (3:1) | N.A. | >300 | 352 |
| 47 | 4-(cyclo-Butyl)piperazin-1-yl | H | H | CH₃— | H | — | N.A. | 122-132 | 349 |
| 48 | 4-(cyclo-Butyl)piperazin-1-yl | H | H | CH₃— | NH₂— | — | N.A. | 132-144 | 364 |
| 49 | 3,3-Dimethylpiperazin-1-yl | H | H | CH₃— | CH₃— | — | N.A. | 110-112 | 337 |
| 50 | 3,3-Dimethylpiperazin-1-yl | H | H | CH₃— | CH₃NH— | — | N.A. | 151-156 | 352 |
| 51 | 3,3-Dimethylpiperazin-1-yl | H | H | c-Propyl- | CH₃— | — | N.A. | N.D. | 363 |
| 52 | 3,3-Dimethylpiperazin-1-yl | H | H | c-Propyl- | NH₂— | — | N.A. | N.D. | 364 |
| 53 | 3,3-Dimethylpiperazin-1-yl | H | H | c-Propyl- | F | — | N.A. | N.D. | 367 |
| 54 | cis-3,5-Dimethylpiperazin-1-yl | H | H | CH₃— | H | HCl (3:1) | N.A. | 343-345 | 323 |
| 55 | cis-3,5-Dimethylpiperazin-1-yl | H | H | CH₃— | CH₃— | — | N.A. | 127-129 | 337 |
| 56 | cis-3,5-Dimethylpiperazin-1-yl | H | H | CH₃— | NH₂— | — | N.A. | 213-215 | 338 |
| 57 | cis-3,5-Dimethylpiperazin-1-yl | H | H | CH₃— | CH₃NH— | — | N.A. | 111-126 | 352 |
| 58 | cis-3,5-Dimethylpiperazin-1-yl | H | H | c-Propyl- | H | — | N.A. | N.D. | 349 |
| 59 | cis-3,5-Dimethylpiperazin-1-yl | H | H | c-Propyl- | NH₂— | — | N.A. | N.D. | 364 |
| 60 | (S)-Hexahydro-pyrrolo[1,2-a]pyrazin-2-yl 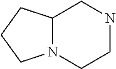 | H | H | CH₃— | H | — | +6 (CH₂Cl₂; C = 1.000) | 154-156 | 335 |
| 61 | (S)-Hexahydro-pyrrolo[1,2-a]pyrazin-2-yl | H | H | CH₃— | CH₃— | — | −1.4 (CH₂Cl₂; | 153-155 | 349 |
| | | | | | | | C = 1.014) | | |
| 62 | (1S,4S)-2,5-diaza-bicyclo[2.2.1]hept-2-yl 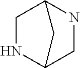 | H | H | CH₃— | H | — | N.D. | 120° C. decomp. | 307 |
| 63 | (R,S)-2,5-diaza-bicyclo[2.2.1]hept-2-yl | H | H | CH₃— | CH₃— | HBr (1:1) | N.A. | >300 | 321 |
| 64 | (R,S)-1,4-Diaza-bicyclo[3.2.2]non-4-yl 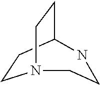 | H | H | c-Propyl- | CH₃— | — | N.A. | N.D. | 375 |
| 65 | (R,S)-Hexahydro-pyrrolo[3,4-b] | H | H | CH₃— | CH₃— | — | N.A. | 131-133 | 335 |
| | pyrrol-5(1H)-yl | | | | | | | | |
| 66 | Hexahydro-pyrrolo[3,4-c]pyrrol-2(1H)-yl 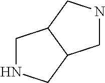 | H | H | CH₃— | H | — | N.A. | 153-155 | 321 |
| 67 | Hexahydro-pyrrolo[3,4-c]pyrrol-2(1H)-yl | H | H | CH₃— | CH₃— | HCl (3:1) | N.A. | 214-221 | 335 |
| 68 | Hexahydro-pyrrolo[3,4-c]pyrrol-2(1H)-yl | H | H | CH₃— | NH₂— | — | N.A. | 155 degrad. | 336 |
| 69 | Hexahydro-pyrrolo[3,4-c]pyrrol-2(1H)-yl | H | H | CH₃— | CH₃NH— | — | N.A. | 169 degrad. | 350 |
| 70 | 5-Methyl-hexahydro-pyrrolo[3,4-c] | H | H | CH₃— | H | — | N.A. | 155-159 | 335 |
| | pyrrol-2(1H)-yl | | | | | | | | |
| 71 | 5-Methyl-hexahydro-pyrrolo[3,4-c] | CH₃— | H | CH₃— | H | — | N.A. | 139-142 | 349 |
| | pyrrol-2(1H)-yl | | | | | | | | |
| 72 | 5-Isopropyl-hexahydro-pyrrolo[3,4-c] | H | H | CH₃— | H | — | CH₃— | 172-175 | 363 |
| | pyrrol-2(1H)-yl | | | | | | | | |
| 73 | 5-Isopropyl-hexahydro-pyrrolo[3,4-c] | H | H | CH₃— | NH₂— | — | N.A. | 215-218 | 378 |
| | pyrrol-2(1H)-yl | | | | | | | | |
| 74 | Hexahydro-pyrrolo[3,4-c]pyrrol-2(1H)-yl | H | H | CH₃— | CH₃O— | — | N.A. | 95 | 351 |
| 75 | (R,S)-Octahydro-pyrrolo[3,4-b]pyridin-6-yl 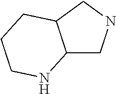 | H | H | CH₃— | H | — | N.A. | 130-135 | 335 |
| 76 | 2,7-Diaza-spiro[3.5]non-7-yl 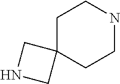 | H | H | CH₃— | CH₃— | — | N.A. | 117-120 | 349 |
| 77 | (R,S)-2,7-Diaza-spiro[4.5]dec-2-yl 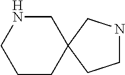 | H | H | CH₃— | H | — | N.A. | 134-136 | 349 |
| 78 | 2,9-Diaza-spiro[5.5]undec-9-yl 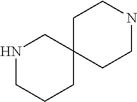 | H | H | CH₃— | H | HCl (3:1) | N.A. | >260 Degrad. | 363 |
| 79 | 2,9-Diaza-spiro[5.5]undec-9-yl 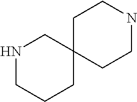 | H | H | CH₃— | CH₃— | HCl (3:1) | N.A. | >295 | 377 |
| 80 | 1-Oxa-4,9-diaza-spiro[5.5]undec-9-yl 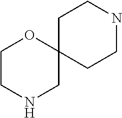 | H | H | CH₃— | H | HCl (3:1) | N.A. | >255 | 365 |
| 81 | 1-Oxa-4,9-diaza-spiro[5.5]undec-9-yl 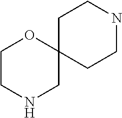 | H | H | CH₃— | NH₂— | HCl (3:1) | N.A. | >300 | 380 |
| 82 | 4-(Pyrrolidin-1-yl)-piperidin-1-yl | H | H | CH₃— | H | — | N.A. | 150-153 | 363 |
| | | | | | | | | 143-145 | |
| 83 | 4-(Pyrrolidin-1-yl)-piperidin-1-yl | H | H | CH₃— | CH₃— | — | N.A. | 124-126 | 377 |
| 84 | 4-(Pyrrolidin-1-yl)-piperidin-1-yl | H | H | CH₃— | NH₂— | — | N.A. | 129-138 | 378 |
| 85 | (R,S)-[1,3′]Bipyrrolidinyl-1′-yl- | H | H | CH₃— | H | — | N.A. | 155-157 | 349 |

### BIOLOGICAL EXAMPLES

The capacity of the compounds of the invention to inhibit the phosphorylation of casein by casein kinase 1 epsilon and delta may be evaluated according to the procedure described in US 2005/0131012.

### Filter-Plate Assay of ATP-³³P for the Screening of CK1 Epsilon Inhibitors:

The effect of the compounds on inhibition of the phosphorylation of casein by the enzyme casein kinase 1 epsilon (CK1 epsilon) is measured, using a casein assay with filtration of ATP-³³P in vitro.

Casein kinase 1 epsilon (0.58 mg/ml) is obtained via fermentation and purification processes performed according to methods that are well known to those skilled in the art, or may also be obtained from Invitrogen Corporation™ (human CK1 epsilon).

The compounds are tested at five different concentrations so as to generate IC₅₀ values, i.e. the concentration at which a compound is capable of inhibiting the enzymatic activity by 50%, or alternatively the percentage of inhibition at a concentration of 10 micromolar.

“U”-bottomed Falcon plates are prepared by placing 5 μl of solutions of the compounds according to the invention at concentrations of 10, 1, 0.1, 0.01 or 0.001 μM in different wells. The solutions of the compounds according to the invention at these various concentrations are prepared by diluting in a test buffer (50 mM Tris, pH 7.5, 10 M MgCl₂, 2 mM DTT and 1 mM EGTA) a stock solution in DMSO at a concentration of 10 mM. Next, 5 μl of dephosphorylated casein are added to a final concentration of 0.2 μg/μl, 20 μl of CK1 epsilon to a final concentration of 3 ng/μl, and 20 μl of ATP-³³P to a final concentration of 0.02 μCi/μl mixed with cold ATP (10 μM final—approximately 2×10⁶ CPM per well). The final total test volume per well is equal to 50 μl.

The “U”-bottomed Falcon® test plate mentioned above is vortexed, and then incubated at ambient temperature for 2 hours. After 2 hours, the reaction is stopped by adding an ice-cold solution of 65 μl of ATP (2 mM) prepared in test buffer.

100 μl of the reaction mixture are then transferred from the “U”-bottomed Falcon® plate into Millipore® MAPH filter plates, preimpregnated with 25 μl of ice-cold 100% TCA.

The Millipore MAPH filter plates are agitated gently and are left to stand at ambient temperature for at least 30 minutes to precipitate the proteins.

After 30 minutes, the filter plates are sequentially washed and filtered with 2×150 μl of 20% TCA, 2×150 μl of 10% TCA and 2×150 μl of 5% TCA (6 washes in total per plate/900 μl per well).

The plates are left to dry overnight at ambient temperature. Next, 40 μl of Microscint-20 Packard® scintillation liquid are added per well and the plates are closed in a leaktight manner. The radiation emitted by each well is then measured for 2 minutes in a Packard® Topcount NXT scintillation counter, in which the values of CPM/well are measured.

The percentage inhibition of the capacity of the enzyme to phosphorylate the substrate (casein) is determined for each concentration of compound tested. These inhibition data expressed as percentages are used to calculate the IC₅₀ value for each compound compared with the controls.

The kinetic studies determined the K_{M} value for ATP as being 21 μM in this test system. Under these conditions, the most active compounds of the invention show IC₅₀ so values (concentration which inhibits 50% of the enzymatic activity of casein kinase 1 epsilon or casein kinase 1 delta) of between 1 nM and 500 nM.

Table 2 below gives the IC₅₀ values for the inhibition of phosphorylation of casein kinase 1 epsilon for a number of compounds according to the invention.

TABLE 2

| Compound | CK1 epsilon Cl₅₀ (nM) |
|---|---|
| 1 | 57 |
| 53 | 292 |
| 66 | 13 |
| 78 | 78 |

The capacity of the compounds of the invention to inhibit the phosphorylation of casein by casein kinase 1 epsilon and delta may be evaluated using a FRET (Fluorescence Resonance Energy Transfer) fluorescence test with the aid of the Z'Lyte™ kinase assay Kit (reference PV3670; Invitrogen Corporation™) according to the manufacturer's instructions.

The casein kinases 1 used are obtained from Invitrogen Corporation (human CK1 epsilon PV3500 and human CK1 delta PV3665).

A peptide substrate, labelled at both ends with a fluorophore donor group (coumarin) and a fluorophore acceptor group (fluorescein), constituting a FRET system is phosphorylated in the presence of ATP by casein kinase 1 epsilon or delta in the presence of increasing concentrations of compounds of the invention.

The mixture is treated with a site-specific protease that specifically cleaves the peptide substrate to form two fluorescent fragments having a large fluorescence emission ratio.

The fluorescence observed is thus related to the capacity of the products of the invention to inhibit the phosphorylation of the peptide substrate by casein kinase 1 epsilon or casein kinase 1 delta.

The compounds of the invention are dissolved at different concentrations starting with a 10 mM stock solution in DMSO diluted in a buffer containing 50 mM HEPS, pH 7.5, 1 m MEGTA, 0.01% Brij-35, 10 mM MgCl for casein kinase 1 epsilon and supplemented with Trizma Base (50 mM), pH 8.0, and NaN₃ (0.01% final) for casein kinase 1 delta.

The phosphorylation of the peptide substrate SER/THR 11 obtained from Invitrogen Corporation™ is performed at a final concentration of 2 μM. The ATP concentration is 4 times the K_{M}, this value being 2 μM for casein kinase 1 epsilon and 4 μM for casein kinase 1 delta.

The emitted fluorescence is measured at wavelengths of 445 and 520 nm (excitation at 400 nm).

Under these conditions, the compounds of the invention that are the most active have IC₅₀ values (concentration that inhibits 50% of the enzymatic activity of casein kinase 1 epsilon or casein kinase 1 delta) of between 1 nM and 500 nM.

Table 3 below gives the IC₅₀ values for the inhibition of phosphorylation of casein kinase 1 delta for a number of compounds according to the invention.

TABLE 3

| Compound | CK1 delta Cl₅₀ (nM) |
|---|---|
| 1 | <1 |
| 54 | <1 |

It is thus seen that the compounds according to the invention have inhibitory activity on the casein kinase 1 epsilon or casein kinase 1 delta enzyme.

### Experimental Protocols for Circadian Cell Assay

Mper1-luc Rat-1 (P2C4) fibroblast cultures were prepared by dividing the cultures every 3-4 days (approximately 10-20% of confluence) on 150 cm² degassed polystyrene tissue culture flasks (Falcon® #35-5001) and maintained in growth medium [EMEM (Cellgro #10-0,0-CV); 10% foetal bovine serum (FBS; Gibco #16000-044); and 50 I.U./ml of penicillin-streptomycin (Cellgro #30-001-CI)] at 37° C. and under 5% CO₂.

Cells obtained from Rat-1 fibroblast cultures at 30-50% of confluence as described above were co-transfected with vectors containing the selection marker for resistance to zeocin for a stable transfection and a luciferase reporter gene controlled by the mPer-1 promoter. After 24 to 48 hours, the cultures were divided on 96-well plates and maintained in growth medium supplemented with 50-100 μg/ml of zeocin (Invitrogen® #45-0430) for 10-14 days. The zeocin-resistant stable transfectants were evaluated for the expression of the reporter by adding 100 μM luciferin (Promega® #E1603®) to the growth medium and by assaying the luciferase activity on a TopCount® scintillation counter (Packard Model #C384V00). The Rat-1 cell clones expressing both zeocin resistance and lucerifase activity controlled by mPer1 were serum-shock synchronized with 50% horse serum [HS (Gibco® #16050-122)] and the activity of the circadian reporter was evaluated. The P2C4 clone of Mper1-luc Rat-1 fibroblasts was selected to test the compound.

Mper1-luc Rat-1 (P2C4) fibroblasts at 40-50% of confluence, obtained according to the protocol described above, were plated out onto 96-well opaque tissue culture plates (Perkin Elmer® #6005680). The cultures are maintained in growth medium supplemented with 100 μg/ml of zeocin (Invitrogen #45-0430) until they reach 100% of confluence (48-72 h). The cultures were then synchronized with 100 μl of synchronization medium [EMEM (Cellgro #10-0,0-CV); 100 I.U./ml of penicillin-streptomycin (Cellgro #30-001-C1); 50% HS (Gibco #16050-122)] for 2 hours at 37° C. and under 5% CO₂. After synchronization, the cultures were rinsed with 100 μl of EMEM (Cellgro #10-010-CV) for 10 minutes at ambient temperature. After rinsing, the medium was replaced with 300 μl of CO₂ independent medium [CO₂I (Gibco #18045-088); 2 mM L-glutamine (Cellgro #25-005-C1); 100 U.I./ml of penicillin-streptomycin (Cellgro #30-001-C1); 100 μM luciferin (Promega #E 1603)]. The compounds of the invention tested for the circadian effects were added to CO₂-independent medium in DMSO at 0.3% (final concentration). The cultures were immediately closed in a leaktight manner with TopSeal-A® film (Packard #6005185) and transferred for the luciferase activity measurement.

After synchronization, the test plates were maintained at 37° C. in a tissue culture incubator (Form a Scientific Model #3914). The in vivo lucerifase activity was estimated by measuring the relative light emission on a TopCount scintillation counter (Packard Model #C384V00).

The period analysis was performed either by determining the interval between the relative light emission minima over several days or by Fourier transform.

The two methods produced a virtually identical period estimation on a range of circadian periods. The power is reported in CE Delta (t+1 h), which is presented as the effective micromolar concentration that induced a 1-hour prolongation of the period. The data were analysed by adjusting a hyperbolic curve to the data expressed as change of period (y-axis) as a function of the concentration of the test compound (x-axis) in the XLfit™ software and the CE Delta (t+1 h) was interpolated from this curve.

Table 4 below gives the CE Delta (t+1 h) for a number of compounds according to the invention.

TABLE 4

| Compound | CE Delta (t + 1 h) (nM) |
|---|---|
| 1 | 20 |
| 53 | 319 |
| 66 | 633 |
| 78 | 515 |

By inhibiting the enzymes CK1epsilon and/or CK1delta, the compounds that are the subjects of the invention modulate the circadian periodicity, and may be useful for treating circadian rhythm-associated disorders.

The compounds according to the invention may in particular be used for the preparation of a medicament for preventing or treating sleep disorders: circadian rhythm disorders, such as, in particular, those caused by jetlag or shift work.

Among the sleep disorders that are especially distinguished are primary sleep disorders such as dyssomnia (for example primary insomnia), parasomnia, hypersomnia (for example excessive somnolence), narcolepsy, sleep disorders related to sleep apnoea, sleep disorders related to the circadian rhythm and otherwise unspecified dyssomnias, sleep disorders associated with medical/psychiatric disorders.

The compounds that are subject-matter of the invention also cause a circadian phase shift and such a property may be useful in the context of a potential monotherapy or combined therapy that is clinically effective in the case of mood disorders.

Among the mood disorders that are especially distinguished are depressive disorders (unipolar depression), bipolar disorders, mood disorders caused by a general medical complaint and also mood disorders induced by pharmacological substances.

Among the bipolar disorders that are especially distinguished are bipolar I disorders and bipolar II disorders, including in particular seasonal affective disorders.

The compounds that are subject-matter of the invention and modulate the circadian periodicity may be useful in the treatment of anxiety and depressive disorders caused in particular by an impairment in the secretion of CRF.

Among the depressive disorders that are especially distinguished are major depressive disorders, dysthymic disorders and otherwise unspecified depressive disorders.

The compounds that are subject-matter of the invention, which modulate the circadian periodicity, may be useful for preparing a medicament for treating diseases related to dependency on abuse substances such as cocaine, morphine, nicotine, ethanol or cannabis.

By inhibiting casein kinase 1 epsilon and/or casein kinase 1 delta, the compounds according to the invention may be used for preparing medicaments, in particular for preparing a medicament for preventing or treating diseases related to hyperphosphorylation of the tau protein, in particular Alzheimer's disease.

These medicaments also find their use in therapy, in particular in the treatment or prevention of diseases caused or exacerbated by the proliferation of cells, in particular tumour cells.

As tumour cell proliferation inhibitors, these compounds are useful in the prevention and treatment of liquid tumours such as leukaemias, solid tumours that are both primary and metastatic, carcinomas and cancers, in particular: breast cancer, lung cancer, small intestine cancer, colorectal cancer; cancer of the respiratory pathways, of the oropharynx and of the hypopharynx; oesophageal cancer; liver cancer, stomach cancer, cancer of the bile ducts, cancer of the gall bladder, pancreatic cancer; cancer of the urinary tracts, including kidney, urothelium and bladder; cancers of the female genital tract, including cancer of the uterus, cervical cancer, ovarian cancer, chloriocarcinomia and trophoblastomia; cancers of the male genital tract, including prostate cancer, cancer of the seminal vesicles, testicular cancer and germinal cell tumours; cancers of the endocrine glands, including thyroid cancer, pituitary cancer and cancer of the adrenal glands; skin cancers, including haemangiomas, melanomas and sarcomas, including Kaposi's sarcoma; brain, nerve, eye or meninges tumours, including astrocytomas, gliomas, glioblastomas, retinoblastomas, neurinomas, neuroblastomas, schwannomas and meningiomas; malignant haematopoietic tumours; leukaemias (Acute Lymphocytic Leukaemia (ALL), Acute Myeloid Leukaemia (AML), Chronic Myeloid Leukaemia (CML), Chronic Lymphocytic Leukaemia (CLL)), chloromas, plasmocytomas, T or B cell leukaemias, Hodgkin or non-Hodgkin lymphomas, myelomas and various malignant homeopathies.

The compounds according to the invention may thus be used for the preparation of medicaments, in particular of medicaments for inhibiting casein kinase 1 epsilon and/or casein kinase 1 delta.

Thus, according to another of its aspects, the invention provides medicaments which comprise a compound of formula (I), or an addition salt thereof with a pharmaceutically acceptable acid, or alternatively a hydrate or a solvate of the compound of formula (I).

According to another of its aspects, the present invention relates to pharmaceutical compositions comprising, as active principle, a compound according to the invention. These pharmaceutical compositions contain an effective dose of at least one compound according to the invention or a pharmaceutically acceptable salt, a hydrate or a solvate of said compound, and also at least one pharmaceutically acceptable excipient.

Said excipients are chosen, according to the pharmaceutical form and the desired mode of administration, from the usual excipients known to a person skilled in the art.

In the pharmaceutical compositions of the present invention for oral, sublingual, subcutaneous, intramuscular, intravenous, topical, local, intratracheal, intranasal, transdermal or rectal administration, the active principle of formula (I) above, or the possible salt, solvate or hydrate thereof, may be administered in unit administration form, as a mixture with standard pharmaceutical excipients, to humans and animals for the prophylaxis or treatment of the above disorders or diseases.

The appropriate unit administration forms include oral-route forms such as tablets, soft or hard gel capsules, powders, granules and oral solutions or suspensions, sublingual, buccal, intratracheal, intraocular and intranasal administration forms, inhalation forms, topical, transdermal, subcutaneous, intramuscular or intravenous administration forms, rectal administration forms and implants. For topical application, the compounds according to the invention may be used in creams, gels, ointments or lotions.

By way of example, a unit administration form of a compound according to the invention in tablet form may comprise the following components:

| | |
|---|---|
| Compound according to the invention | 50.0 mg |
| Mannitol | 223.75 mg |
| Sodium croscaramellose | 6.0 mg |
| Corn starch | 15.0 mg |
| Hydroxypropylmethylcellulose | 2.25 mg |
| Magnesium stearate | 3.0 mg |

Via the oral route, the dose of active principle administered per day may reach from 0.1 to mg/kg, in one or more dosage intakes.

There may be particular cases in which higher or lower dosages are appropriate; such dosages do not depart from the context of the invention. According to the usual practice, the dosage that is appropriate to each patient is determined by the practitioner according to the mode of administration and the weight and response of said patient.

According to another of its aspects, the present invention also relates to a method for treating the pathologies indicated above, which comprises the administration to a patient of an effective dose of a compound according to the invention, or a pharmaceutically acceptable salt or hydrate or solvate thereof.

1. Compound conforming to the general formula (I) 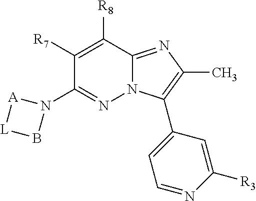
in which
R₂ represents a C₁₋₆-alkyl, C₃₋₇-cycloalkyl, C₃₋₇-cycloalkyl-C₁₋₄-alkyl, C₁₋₄-alkyloxy-C₁₋₄-alkyl, C₃₋₇-cycloalkyloxy-C₁₋₄-alkyl, C₃₋₇-cycloalkyl-C₁₋₄-alkyloxy-C₁₋₄-alkyl, hydroxy-C₁₋₆-alkyl or C₁₋₄-fluoroalkyl group;
R₃ represents a hydrogen atom or a substituent selected from halogen atoms and C₁₋₃-alkyl, —NR₄R₅, hydroxyl or C₁₋₄-alkyloxy groups;
A represents a C₁₋₇-alkylene group optionally substituted by one or two groups Rₐ;
B represents a C₁₋₇-alkylene group optionally substituted by a group R_{b};
L represents either a nitrogen atom optionally substituted by a group R or R_{d}, or a carbon atom substituted by a group Rₑ₁ and a group R_{d} or two groups Rₑ₂;
the carbon atoms of A and of B being optionally substituted by one or more groups R_{f} that are identical or different from one another;
Rₐ, R_{b} and Rₑ are defined such that:
two groups Rₐ may together form a C₁₋₆-alkylene group;
Rₐ and R_{b} may together form a bond or a C₁₋₆-alkylene group;
Rₐ and R_{c} may together form a bond or a C₁₋₆-alkylene group;
R_{b} and R, may together form a bond or a C₁₋₆-alkylene group;
R_{d} represents a group selected from the hydrogen atom and C₁₋₆-alkyl, C₃₋₇-cycloalkyl, C₃₋₇-cycloalkyl-C₁₋₆-alkyl, C₁₋₆-alkylthio-C₁₋₆-alkyl, C₁₋₆-alkyloxy-C₁₋₆-alkyl, C₁₋₆-fluoroalkyl and hydroxy-C₁₋₆-alkyl groups;
Rₑ₁ represents a group —NR₄R₅or a cyclic monoamine optionally containing an oxygen atom, the cyclic monoamine being optionally substituted by one or more substituents selected from the fluorine atom and C₁₋₆-alkyl, C₁₋₆-alkyloxy and hydroxyl groups;
two radicals Rₑ₂ form, with the carbon atom which carries them, a cyclic monoamine optionally containing an oxygen atom, this cyclic monoamine being optionally substituted by one or more groups R_{f} that are identical or different from one another;
R_{f} represents a C₁₋₆-alkyl, C₃₋₇-cycloalkyl, C₃₋₇-cycloalkyl-C₁₋₆-alkyl, C₁₋₆-alkyloxy-C₁₋₆-alkyl, C₃₋₇-cycloalkyloxy-C₁₋₄-alkyl, C₃₋₇-cycloalkyl-C₁₋₄-alkyloxy-C₁₋₄-alkyl, hydroxy-C₁₋₆-alkyl or C₁₋₆-fluoroalkyl group;
R₄ and R₅ represent, independently of one another, a hydrogen atom or a C₁₋₄-alkyl, C₃₋₇-cycloalkyl or C₃₋₇-cycloalkyl-C₆-alkyl group;
R₇ and R₈ represent, independently of one another, a hydrogen atom or a C₁₋₆-alkyl group; in the form of a base or acid addition salt.

2. Compound of general formula (I) according to claim 1, characterized in that:
R₂ represents a C₁₋₄-alkyl, C₃₋₄-cycloalkyl-C₁₋₄-alkyl, C₁₋₄-alkyloxy-C₁₋₄-alkyl or C₁₋₄-fluoroalkyl group.

3. Compound of general formula (I) according to claim 1, characterized in that:
R₃ represents hydrogen, fluorine or chlorine atom or a methyl, methylamino, —NH₂ or methoxy group.

4. Compound of general formula (I) according to claim 1, characterized in that:
R₇ and R₈ represent, independently of one another, a hydrogen atom or a methyl group.

5. Compound of general formula (I) according to claim 1, characterized in that:
A represents a C₁₋₇-alkylene group optionally substituted by one or two groups Rₐ;
B represents a C₁₋₇-alkylene group optionally substituted by a group R_{b};
L represents a nitrogen atom optionally substituted by a group R^{c} or R_{d},
the carbon atoms of A and of B being optionally substituted by one or more groups R_{f} that are identical or different from one another;
two groups Rₐ may together form a C₁₋₆-alkylene group;
Rₐ and R_{b} may together form a bond or a C₁₋₆-alkylene group;
Rₐ and R_{c} may together form a bond or a C₁₋₆-alkylene group;
R_{b} and R_{c} may together form a bond or a C₁₋₆-alkylene group;
R_{d} represents a group selected from the hydrogen atom and C₁₋₆-alkyl, C₃₋₇-cycloalkyl, C₃₋₇-cycloalkyl-C₁₋₆-alkyl, C₁₋₆-alkylthio-C₁₋₆-alkyl, C₁₋₆-alkyloxy-C₁₋₆-alkyl, C₁₋₆-fluoroalkyl and hydroxy-C₁₋₆-alkyl groups;
R_{f} represents a C₁₋₆-alkyl, C₃₋₇-cycloalkyl, C₁₋₆-alkyloxy-C₁₋₆-alkyl, C₃₋₇-cycloalkyloxy-C₁₋₄-alkyl, C₃₋₇-cycloalkyl-C₁₋₄-alkyloxy-C₁₋₄-alkyl or hydroxy-C₁₋₆-alkyl group.

6. Compound of general formula (I) according to claim 1, characterized in that:
A represents a C₁₋₇-alkylene group optionally substituted by one or two groups Rₐ;
B represents a C₁₋₇-alkylene group optionally substituted by a group R_{b};
L represents a carbon atom substituted by two groups Rₑ₂;
the carbon atoms of A and of B being optionally substituted by one or more groups R_{f} that are identical or different from one another;
two radicals Rₑ₂ form, with the carbon atom which carries them, a cyclic monoamine optionally containing an oxygen atom, this cyclic monoamine being optionally substituted by one or more groups R_{f} that are identical or different from one another;
R_{f} represents a C₁₋₆-alkyl group.

7. Compound of general formula (I) according to claim 1, characterized in that:
A represents a C₁₋₇-alkylene group;
B represents a C₁₋₇-alkylene group;
L represents a carbon atom substituted by a group Rₑ₁ and a group R_{d};
R_{d} represents a hydrogen atom;
Rₑ₁ represents a group —NR₄R₅ in which R₄ and R₅ represent, independently of one another, a hydrogen atom or a C₁₋₄-alkyl group, or else Rₑ₁ represents a cyclic monoamine optionally containing an oxygen atom, the cyclic monoamine being optionally substituted by one or more substituents selected from C₁₋₆-alkyl and hydroxyl groups.

8. Compound of general formula (I) according to claim 1, characterized in that:
R₂ represents a methyl, ethyl, isopropyl, isobutyl, cyclopropyl, cyclobutyl, cyclopropylmethyl, methoxymethyl or trifluoromethyl group;
R₃ represents hydrogen, fluorine or chlorine atom or a methyl, methylamino, —NH₂ or methoxy group;
the cyclic amine formed by —N-A-L-B— represents a piperazin-1-yl, (R,S)-3-methylpiperazin-1-yl, (R)-3-methylpiperazin-1-yl, (S)-3-methylpiperazin-1-yl, 4-methylpiperazin-1-yl, 4-ethyl-piperazin-1-yl, 4-(isopropyl)piperazin-1-yl, 4-(cyclobutyl)piperazin-1-yl, (R,S)-3-(hydroxymethyl)piperazin-1-yl, 3,3-dimethylpiperazin-1-yl, cis-3,5-dimethylpiperazin-1-yl, (S)-hexahydropyrrolo[1,2-a]pyrazin-2-yl, (1S,4S)-2,5-diazabicyclo[2.2.1]hept-2-yl, (R,S)-2,5-diazabicyclo[2.2.1]hept-2-yl, (R,S)-1,4-diazabicyclonon-4-yl, (R,S)-hexahydropyrrolo[3,4-b]pyrrol-5(1H)-yl, hexahydropyrrolo[3,4-c]pyrrol-2(1H)-yl, 5-methylhexahydropyrrolo[3,4-c]pyrrol-2(1H)-yl, 5-isopropylhexahydropyrrolo[3,4-c]pyrrol-2(1H)-yl or (R,S)-octahydropyrrolo[3,4-b]pyridin-6-yl group;
R₇ and R₈ represent, independently of one another, a hydrogen atom or a methyl group.

9. Compound of general formula (I) according to claim 1, characterized in that:
R₂ represents a methyl group;
R₃ represents hydrogen atom;
the cyclic amine formed by —N-A-L-B— represents a 2,7-diazaspiro[3.5]non-7-yl, (R,S)-diazaspiro[4.5]dec-2-yl, 2,9-diazaspiro[5.5]undec-9-yl or 1-oxa-4,9-diazaspiroundec-9-yl;
R₇ and R₈ represent, independently of one another, a hydrogen atom.

10. Compound of general formula (I) according to claim 1, characterized in that:
R₂ represents a methyl group;
R₃ represents hydrogen atom or a methyl group;
the cyclic amine formed by —N-A-L-B— represents a 4-(pyrrolidin-1-yl)piperidin-1-yl or an (R,S)-[1,3′]bipyrrolidinyl-1′-yl;
R₇ and R₈ represent, independently of one another, a hydrogen atom.

11. Compound of general formula (I) according to claim 1, selected from:
2-Methyl-6-piperazin-1-yl-3-pyridin-4-yl-imidazo[1,2-b]pyridazine and its hydrochloride (3:1);
3-(2-Fluoropyridin-4-yl)-2-methyl-6-piperazin-1-ylimidazo[1,2-b]pyridazine;
2,7,8-Trimethyl-6-piperazin-1-yl-3-pyridin-4-ylimidazo[1,2-b]pyridazine and its hydrochloride (3:1);
2-Methoxymethyl-6-piperazin-1-yl-3-pyridin-4-ylimidazo[1,2-b]pyridazine;
2-Ethyl-6-piperazin-1-yl-3-pyridin-4-yl-imidazo[1,2-b]pyridazine and its hydrochloride (3:1);
2-Ethyl-6-piperazin-1-yl-3-(2-methylpyridin-4-yl)imidazo[1,2-b]pyridazine;
2-Isopropyl-6-piperazin-1-yl-3-pyridin-4-ylimidazo[1,2-b]pyridazine;
2-Isopropyl-3-(2-methylpyridin-4-yl)-6-piperazin-1-ylimidazo[1,2-b]pyridazine;
2-Cyclopropyl-6-piperazin-1-yl-3-pyridin-4-ylimidazo[1,2-b]pyridazine;
2-Cyclopropyl-3-(2-methylpyridin-4-yl)-6-piperazin-1-ylimidazo[1,2-b]pyridazine;
4-(2-Cyclopropyl-6-piperazin-1-ylimidazo[1,2-b]pyridazin-3-yl)pyridin-2-ylamine;
2-Cyclopropyl-3-(2-methoxypyridin-4-yl)-6-piperazin-1-ylimidazo[1,2-b]pyridazine;
3-(2-Chloropyridin-4-yl)-2-cyclopropyl-6-piperazin-1-ylimidazo[1,2-b]pyridazine;
2-Isobutyl-6-piperazin-1-yl-3-pyridin-4-ylimidazo[1,2-b]pyridazine and its hydrochloride (3:1);
2-Cyclopropylmethyl-6-piperazin-1-yl-3-pyridin-4-ylimidazo[1,2-b]pyridazine;
2-Cyclobutyl-6-piperazin-1-yl-3-pyridin-4-ylimidazo[1,2-b]pyridazine;
(R,S)-2-Methyl-6-(3-methylpiperazin-1-yl)-3-pyridin-4-ylimidazo[1,2-b]pyridazine;
(R,S)-2-Methyl-6-(3-methylpiperazin-1-yl)-3-(2-methylpyridin-4-yl)imidazo[1,2-b]pyridazine;
(R,S)-2-Cyclopropyl-6-(3-methylpiperazin-1-yl)-3-(2-methylpyridin-4-yl)imidazo[1,2-b]-pyridazine;
(R,S)-4-[2-Cyclopropyl-6-(3-methylpiperazin-1-yl)imidazo[1,2-b]pyridazin-3-yl]pyridin-2-ylamine;
(R,S)-2-Cyclopropyl-3-(2-methoxypyridin-4-yl)-6-(3-methylpiperazin-1-yl)imidazo[1,2-b]-pyridazine;
(R,S)-3-(2-Chloropyridin-4-yl)-2-cyclopropyl-6-(3-methylpiperazin-1-yl)imidazo[1,2-b]-pyridazine;
2-Methyl-6-((R)-3-methylpiperazin-1-yl)-3-pyridin-4-ylimidazo[1,2-b]pyridazine;
2-Methyl-6-((R)-3-methylpiperazin-1-yl)-3-(2-methylpyridin-4-yl)imidazo[1,2-b]pyridazine;
4-[2-Methyl-6-((R)-3-methylpiperazin-1-yl)imidazo[1,2-b]pyridazin-3-yl]pyridin-2-ylamine;
Methyl-{4-[2-methyl-6-((R)-3-methylpiperazin-1-yl)imidazo[1,2-b]pyridazin-3-yl]pyridin-2-yl}-amine;
3-(2-Methoxypyridin-4-yl)-2-methyl-6-((R)-3-methylpiperazin-1-yl)imidazo[1,2-b]pyridazine;
2-Ethyl-6-((R)-3-methylpiperazin-1-yl)-3-pyridin-4-ylimidazo[1,2-b]pyridazine;
2-Ethyl-3-(2-fluoropyridin-4-yl)-6-((R)-3-methylpiperazin-1-yl)imidazo[1,2-b]pyridazine;
2-Cyclopropylmethyl-6-((R)-3-methylpiperazin-1-yl)-3-pyridin-4-ylimidazo[1,2-b]pyridazine;
2-Cyclopropylmethyl-6-((R)-3-methylpiperazin-1-yl)-3-(2-methylpyridin-4-yl)imidazo[1,2-b]pyridazine;
2-Methyl-6-((S)-3-methylpiperazin-1-yl)-3-pyridin-4-ylimidazo[1,2-b]pyridazine;
3-(2-Methoxypyridin-4-yl)-2-methyl-6-((S)-3-methylpiperazin-1-yl)imidazo[1,2-b]pyridazine;
2-Cyclopropyl-6-((S)-3-methylpiperazin-1-yl)-3-pyridin-4-ylimidazo[1,2-b]pyridazine;
4-[2-Methyl-6-((S)-3-methylpiperazin-1-yl)imidazo[1,2-b]pyridazin-3-yl]pyridin-2-ylamine;
2-Methyl-6-(4-methylpiperazin-1-yl)-3-pyridin-4-ylimidazo[1,2-b]pyridazine;
6-(4-Methylpiperazin-1-yl)-3-pyridin-4-yl-2-trifluoromethylimidazo[1,2-b]pyridazine;
[4-(2-Methyl-3-pyridin-4-ylimidazo[1,2-b]pyridazin-6-yl)piperazin-2-yl]methanol;
6-(4-Ethylpiperazin-1-yl)-2-methyl-3-pyridin-4-ylimidazo[1,2-b]pyridazine and its hydrochloride (3:1);
6-(4-Ethylpiperazin-1-yl)-2-methyl-3-(2-methylpyridin-4-yl)imidazo[1,2-b]pyridazine;
6-(4-isopropylpiperazin-1-yl)-2-methyl-3-pyridin-4-ylimidazo[1,2-b]pyridazine and its hydrochloride (3:1);
6-(4-Isopropylpiperazin-1-yl)-2-methyl-3-(2-methylpyridin-4-yl)imidazo[1,2-b]pyridazine;
4-[6-(4-Isopropylpiperazin-1-yl)-2-methylimidazo[1,2-b]pyridazin-3-yl]pyridin-2-yl}-amine and its hydrochloride (3:1);
6-(4-Cyclobutylpiperazin-1-yl)-2-methyl-3-pyridin-4-ylimidazo[1,2-b]pyridazine;
4-[6-(4-Cyclobutylpiperazin-1-yl)-2-methylimidazo[1,2-b]pyridazin-3-yl]pyridin-2-ylamine;
6-(3,3-Dimethylpiperazin-1-yl)-2-methyl-3-(2-methylpyridin-4-yl)imidazo[1,2-b]pyridazine;
{4-[6-(3,3-Dimethylpiperazin-1-yl)-2-methylimidazo[1,2-b]pyridazin-3-yl]pyridin-2-yl}methyl-amine;
2-Cyclopropyl-6-(3,3-dimethylpiperazin-1-yl)-3-(2-methylpyridin-4-yl)imidazo[1,2-b]-pyridazine;
4-[2-Cyclopropyl-6-(3,3-dimethylpiperazin-1-yl)imidazo[1,2-b]pyridazin-3-yl]pyridin-2-ylamine;
2-Cyclopropyl-6-(3,3-dimethylpiperazin-1-yl)-3-(2-fluoropyridin-4-yl)imidazo[1,2-b]pyridazine;
6-(cis-3,5-Dimethylpiperazin-1-yl)-2-methyl-3-pyridin-4-ylimidazo[1,2-b]pyridazine and its hydrochloride (3:1);
6-(cis-3,5-Dimethylpiperazin-1-yl)-2-methyl-3-(2-methylpyridin-4-yl)imidazo[1,2-b]pyridazine;
4-[6-(cis-3,5-Dimethylpiperazin-1-yl)-2-methylimidazo[1,2-b]pyridazin-3-yl]pyridin-2-ylamine;
{4-[6-(cis-3,5-Dimethylpiperazin-1-yl)-2-methylimidazo[1,2-b]pyridazin-3-yl]pyridin-2-yl}-methylamine;
2-Cyclopropyl-6-(cis-3,5-dimethylpiperazin-1-yl)-3-=pyridin-4-ylimidazo[1,2-b]pyridazine;
4-[2-Cyclopropyl-6-(cis-3,5-dimethylpiperazin-1-yl)imidazo[1,2-b]pyridazin-3-yl]pyridin-2-ylamine;
6-(S)-Hexahydropyrrolo[1,2-a]pyrazin-2-yl-2-methyl-3-pyridin-4-ylimidazo[1,2-b]pyridazine;
6-(S)-Hexahydropyrrolo[1,2-a]pyrazin-2-yl-2-methyl-3-(2-methylpyridin-4-yl)imidazo[1,2-b]pyridazine;
6-(1S,4S)-2,5-Diazabicyclo[2.2.1]hept-2-yl-2-methyl-3-pyridin-4-ylimidazo[1,2-b]pyridazine;
(R,S)-6-(2,5-Diazabicyclo[2.2.1]hept-2-yl)-2-methyl-3-(2-methylpyridin-4-yl)imidazo[1,2-b]pyridazine and its hydrobromide (1:1);
4-[2-Cyclopropyl-6-(1,4-dazabicyclo[3.2.2]non-4-yl)-3-(2-methylpyridin-4-yl)imidazo[1,2-b]pyridazine;
(R,S)-6-(Hexahydropyrrolo[3,4-b]pyrrol-5(1H-yl)-2-methyl-3-(2-methylpyridin-4-yl)imidazo[1,2-b]pyridazine;
6-Hexahydropyrrolo[3,4-c]pyrrol-2 (1H)-yl-2-methyl-3-pyridin-4-ylimidazo[1,2-b]pyridazine;
6-Hexahydropyrrolo[3,4-c]pyrrol-2(1H)-yl-2-methyl-3-(2-methylpyridin-4-yl)-imidazo[1,2-b]pyridazine and its hydrochloride (3:1);
4-(6-Hexahydropyrrolo[3,4-c]pyrrol-2(1H)-yl-2-methylimidazo[1,2-b]pyridazin-3-yl)pyridin-2-ylamine;
[4-(6-Hexahydropyrrolo[3,4-c]pyrrol-2(1H)-yl-2-methylimidazo[1,2-b]pyridazin-3-yl)pyridin-2-yl]-methylamine;
2-Methyl-6-(5-methylhexahydropyrrolo[3,4-c]pyrrol-2(1H)-yl)-3-pyridin-4-ylimidazo[1,2-b]pyridazine;
2,7-Dimethyl-6-(5-methylhexahydropyrrolo[3,4-c]pyrrol-2(II)-yl)-3-pyridin-4-ylimidazo[1,2-b]pyridazine;
6-(−5-Isopropylhexahydropyrrolo[3,4-c]pyrrol-2(1H)-yl)-2-methyl-3-pyridin-4-ylimidazo[1,2-b]pyridazine;
4-[6-(−5-Isopropylhexahydropyrrolo[3,4-c]pyrrol-2(1H)-yl)-2-methylimidazo[1,2-b]pyridazin-3-yl]pyridin-2-ylamine;
6-Hexahydropyrrolo[3,4-c]pyrrol-2(1H)-yl-3-(2-methoxypyridin-4-yl)-2-methylimidazo[1,2-b]pyridazine;
(R,S)-2-Methyl-6-(octahydropyrrolo[3,4-b]pyridin-6-yl)-3-pyridin-4-ylimidazo[1,2-b]-pyridazine;
6-(2,7-Diazaspiro[3.5]non-7-yl)-2-methyl-3-(2-methylpyridin-4-yl)imidazo[1,2-b]pyridazine;
(R,S)-6-(2,7-Diazaspiro[4.5]dec-2-yl)-2-methyl-3-pyridin-4-ylimidazo[1,2-b]pyridazine;
9-(2-Methyl-3-pyridin-4-yl-imidazo[1,2-b]pyridazin-6-yl)-2,9-diazaspiro[5.5]undecane and its hydrochloride (3:1);
9-[2-Methyl-3-(2-methylpyridin-4-yl)imidazo[1,2-b]pyridazin-6-yl]-2,9-diazaspiro[5.5]-undecane and its hydrochloride (3:1);
9-(2-Methyl-3-pyridin-4-ylimidazo[1,2-b]pyridazin-6-yl)-1-oxa-4,9-diazaspiro[5.5]undecane and its hydrochloride (3:1);
4-[2-Methyl-6-(1-oxa-4,9-diazaspiro[5.5]undec-9-yl)imidazo[1,2-b]pyridazin-3-yl]pyridin-2-ylamine and its hydrochloride (3:1);
2-Methyl-3-pyridin-4-yl-6-(4-pyrrolidin-1-ylpiperidin-1-yl)imidazo[1,2-b]pyridazine;
2-Methyl-3-(2-methylpyridin-4-yl)-6-(4-pyrrolidin-1 ylpiperidin-1-yl)imidazo[1,2-b]pyridazine;
4-[2-Methyl-6-(4-pyrrolidin-1-ylpiperidin-1-yl)imidazo[1,2-b]pyridazin-3-yl]pyridin-2-ylamine;
(R,S)-6-[1,3′]Bipyrrolidinyl-1′-yl-2-methyl-3-pyridin-4-ylimidazo[1,2-b]pyridazine.

12. Process for preparing a compound of formula (I) according to claim 1, characterized in that a compound of general formula (IIa) 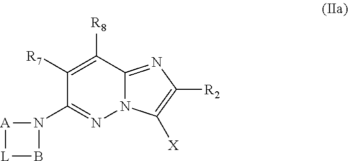
in which R₂, A, L, B, R₇ and R₈ are as defined according to claim 1 and X represents a bromine or iodine atom is reacted with a compound of formula (IVa) 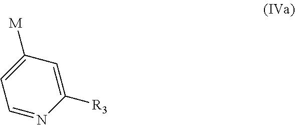
in which R₃ is as defined in claim 1 and M represents a trialkylstannyl, dihydroxyboryl or dialkoxyboryl group.

13. Process for preparing a compound of formula (I) according to claim 1, characterized in that.
a) a compound of general formula (II) 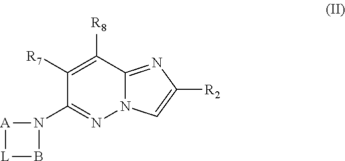
in which R₂, A, L, B, R₇ and R₈ are as defined according to claim 1 is reacted with a mixture of a pyridine derivative of general formula (IVb) 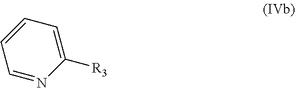
in which R₃ represents a hydrogen atom or a C₁₋₃-alkyl group in the presence of alkyl chloroformate, to give a compound of formula (IIb) 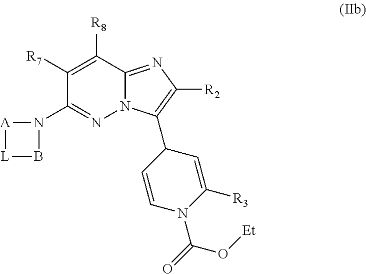
in which R₂, A, L, B, R₇ and R₈ are as defined according to claim 1 and R₃ represents a hydrogen atom or a C₁₋₃-alkyl group; and
b) the compound of general formula (IIb) obtained in step a) is reacted with orthochloranil in a solvent.

14. Process for preparing a compound of formula (I) according to claim 1, characterized in that a compound of general formula (II) 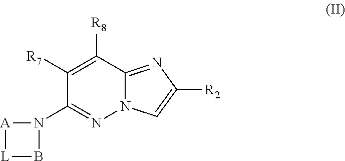
in which R₂, R₇, R₈, A, L and B are as defined according to claim 1, is reacted with a compound of general formula (IVc) 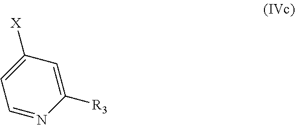
in which R₃ is as defined according to claim 1 and X represents a halogen atom, in the presence of a catalyst and of an inorganic base and in an aprotic polar solvent.

15. A pharmaceutical composition comprising the compound of claim 1, in the form of a base or an addition salt with a pharmaceutically acceptable acid.

16. The pharmaceutical composition of claim 15 further comprising, at least one pharmaceutically acceptable excipient.

17. A method of treating or preventing sleep disorders or circadian rhythm disorders in a patient in need thereof comprising administering to said patient a therapeutically effective amount of the pharmaceutical composition of claim 15.

18. A method of treating or preventing bipolar disorders in a patient in need thereof comprising administering to said patient a therapeutically effective amount of the pharmaceutical composition of claim 15.

19. A method of treating or preventing diseases associated with a dependence on abuse substances in a patient in need thereof comprising administering to said patient a therapeutically effective amount of the pharmaceutical composition of claim 15.

20. A method of treating or preventing diseases related to hyperphosphorylation of the tau protein in a patient in need thereof comprising administering to said patient a therapeutically effective amount of the pharmaceutical composition of claim 15.

21. A method of treating or preventing diseases caused or exacerbated by cell proliferation in a patient in need thereof comprising administering to said patient a therapeutically effective amount of the pharmaceutical composition of claim 15.

22. The method according to claim 21, characterized in that the cells are tumour cells.
